(19) United States
(12) Patent Application Publication
(10) Pub. No.: US 2022/0204449 A1
(43) Pub. Date: Jun. 30, 2022

(54) BIASED AGONISTS OF OPIOID RECEPTORS

(71) Applicant: ILDONG PHARMACEUTICAL CO., LTD., Seoul (KR)

(72) Inventors: Woojin JEON, Seoul (KR); Do Sup LEE, Seoul (KR); Suh-Youn SHON, Seoul (KR); Junsu BAN, Seoul (KR); Sungjun HONG, Seoul (KR); Jin Hee LEE, Seoul (KR); Jaeho HEO, Seoul (KR); Jungwoo LEE, Seoul (KR); JoonTae PARK, Seoul (KR); Kyung Mi AN, Seoul (KR); Hongchul YOON, Seoul (KR)

(21) Appl. No.: 17/548,288

(22) Filed: Dec. 10, 2021

Related U.S. Application Data
(60) Provisional application No. 63/124,602, filed on Dec. 11, 2020.

(51) Int. Cl. C07D 209/08; C07D 307/79; C07D 319/18

(52) U.S. Cl. CPC C07D 209/08; C07B 2200/07; C07D 319/18; C07D 307/79

(57) ABSTRACT

The present disclosure provides pharmaceutical compositions and compounds having dual agonist activity at the μ and δ opioid receptors. The agonist compounds of this disclosure can also provide GPCR functional selectivity, including selective activity in the Gi pathway. In some embodiments, the compounds of this disclosure are biased μ-opioid and/or δ-opioid receptor agonists that do not significantly recruit β-arrestins, but can activate G-protein-dependent pathways, and thus can be administered without risk of significant undesirable side effects exhibited by conventional opioid receptor agonists. In some embodiments there is provided a substantially optically pure compound having dual agonist activity at the μ and δ opioid receptors.

### 1. CROSS REFERENCE TO RELATED APPLICATIONS

This application claims the benefit of U.S. Provisional Application No. 63/124,602, filed Dec. 11, 2020, which is hereby incorporated in its entirety by reference.

### 2. BACKGROUND OF THE INVENTION

Chronic pain is a common and significant public health challenge that has a great impact on quality of life of patients. Pain treatments in use include both opioids and non-opioid analgesics. Opiates are potent analgesics, but their clinical use can be limited by undesirable side effects.

Opiate addiction, coupled with the potentially lethal side effects of opiates like respiratory depression, has driven the development of safer and more effective analgesics. Morphine and codeine are more reliably effective analgesics than raw opium but retained its undesirable side effects. The classification of opioid receptors into μ, δ, and K subtypes lead to the investigation of subtype-specific molecules that might escape the liabilities of morphinan-based opiates. Potent synthetic opioid agonists such as methadone and fentanyl, and endogenous opioid peptides, were developed as analgesics, but elimination of their undesirable side effects has remained elusive.

G protein-coupled receptors (GPCRs) regulate a wide variety of important cellular processes and are targets of many approved drugs. GPCRs signal by activating heterotrimeric G proteins and must couple to a select subset of G proteins to produce appropriate intracellular responses. The μ (mu) opioid receptor (μ-OR) is a molecular target for opiate-mediated analgesia. Opioid-induced analgesia can result from μ-opioid receptor (μOR) signaling through the G protein Gi, while many side effects, including respiratory depression and constipation, may be conferred via β-arrestin pathway signaling.

Agonists specific to the μOR and biased toward the inhibitory G protein (Gi) signaling pathway are therefore sought both as therapeutic leads and as molecular probes of μOR signaling. A biased μ-opioid receptor agonist is an agonist that does not significantly recruit β-arrestins, but can activate G-protein-dependent pathways.

### 3. SUMMARY OF THE INVENTION

The present disclosure provides pharmaceutical compositions and compounds having dual agonist activity at the μ and δ opioid receptors. The agonist compounds of this disclosure can also provide GPCR functional selectivity, including selective activity in the Gi pathway. In some embodiments, the compounds of this disclosure are biased μ-opioid and/or δ-opioid receptor agonists that do not significantly recruit β-arrestins, but can activate G-protein-dependent pathways, and thus can be administered without risk of significant undesirable side effects exhibited by conventional opioid receptor agonists. In some embodiments there is provided a substantially optically pure compound having dual agonist activity at the μ and δ opioid receptors.

In some embodiments, the present disclosure provides a substantially optically pure (+) stereoisomer of a subject compound.

In some embodiments, the present disclosure provides a substantially optically pure (−) stereoisomer of a subject compound.

In some embodiments, the present disclosure provides a pharmaceutical composition comprising:
(i) a compound of formula (I):

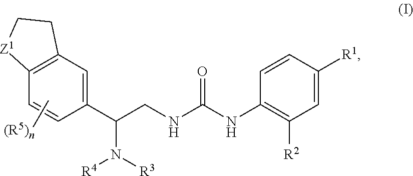

or a stereoisomer thereof, or a pharmaceutically acceptable salt thereof,
wherein:

Z¹ is selected from —O— and —NR⁶—, wherein R⁶ is selected from —H and optionally substituted (C₁-C₃)alkyl;

R¹, R² and each R⁵ are independently selected from H, halogen, OH, (C₁-C₃)alkyl, substituted (C₁-C₃)alkyl, (C₁-C₃)alkoxy, substituted (C₁-C₃)alkoxy; and

R³ and R⁴ are independently selected from —H and optionally substituted (C₁-C₃)alkyl; and

n is 0 to 3; and

(ii) a pharmaceutically acceptable excipient.

In some embodiments of the pharmaceutical composition, the compound is:

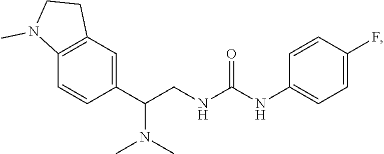 [SMILES: CN1CCc2cc(C(CNC(=O)Nc3ccc(F)cc3)N(C)C)ccc21]

or a substantially optically pure stereoisomer thereof, or a pharmaceutically acceptable salt thereof.

In some embodiments of the pharmaceutical composition, the compound is:

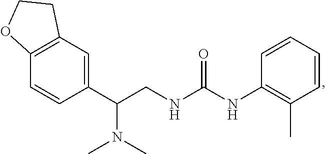 [SMILES: Cc1ccccc1NC(=O)NCC(c1ccc2c(c1)CCO2)N(C)C]

or a substantially optically pure stereoisomer thereof, or a pharmaceutically acceptable salt thereof.

In some embodiments, the present disclosure provides a pharmaceutical composition comprising:

(i) a compound of formula (II):

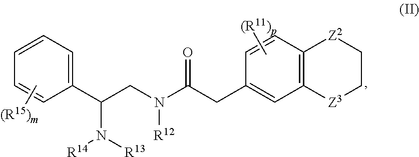

or a stereoisomer thereof, or a pharmaceutically acceptable salt thereof,
wherein:

Z² and Z³ are independently selected from —O— and —NR¹⁶—, wherein R¹⁶ is selected from —H and optionally substituted (C₁-C₃)alkyl;

R¹² is selected from —H and optionally substituted (C₁-C₃)alkyl; and

each R¹¹ and each R¹⁵ are independently selected from —H, -halogen, —OH, —CF₃, —OCF₃, —CN, —NH₂, —NO₂, optionally substituted (C₁-C₆)alkyl, optionally substituted (C₁-C₆)alkoxy, —C(O)OR¹², —C(O)NHR¹², —SO₂NHR¹², —NR¹³R¹⁴, —NHC(O)R¹², and —SO₃H;

m is 0 to 5; and

p is 0 to 3; and

(ii) a pharmaceutically acceptable excipient.

In some embodiments of the pharmaceutical composition, the compound of formula (II) is a compound of formula (III):

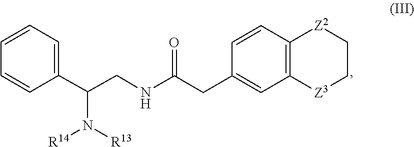

or a stereoisomer thereof, or a pharmaceutically acceptable salt thereof.

In some embodiments of the pharmaceutical composition, the compound of formula (III) is

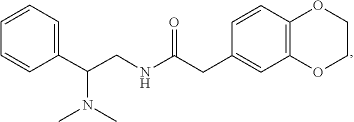 [SMILES: CN(C)C(CNC(=O)Cc1ccc2c(c1)OCCO2)c1ccccc1]

or a substantially optically pure stereoisomer thereof, or a pharmaceutically acceptable salt thereof.

In some embodiments, the present disclosure provides a method of modulating the activity of an opioid receptor, comprising contacting a biological system with a substantially optically pure stereoisomer or a pharmaceutical composition as described herein.

In some embodiments, the present disclosure provides a method of treating pain, comprising administering to a subject having pain an effective amount of a pharmaceutical composition as described herein.

### 4. BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 shows exemplary graphs of dominant assay results that exhibit the effects of decreasing receptor number on two agonists.

FIGS. 2A-2F shows the results from a dominant assay using OPRM cell lines. DAMGO is a full agonist of OPRM (FIG. 2A). The maximal response of TRV130 (FIG. 2B) and PZM21 (FIG. 2C) are susceptible to decreasing receptor populations than DAMGO. However, compound 1 (FIG. 2D), compound 2 (FIG. 2E), and compound 3 (FIG. 2F) showed a similar pattern as compared to DAMGO.

FIG. 3A-3F shows the results from a dominant assay using OPRD cell lines. SNC80 is a full agonist for OPRD (FIG. 3A). The maximal responses of TRV130 (FIG. 3B), PZM21 (FIG. 3C), and exemplary compounds 1-3 (FIGS. 3D-3F, respectively) were more susceptible to decreasing receptor populations than SNC80.

FIG. 4 shows β-arrestin assay result (% bARR2 response) for compounds 4-9 on the μ-Opioid receptor (OPRM) vs a full agonist of OPRM, DAMGO and as compared to morphine.

FIG. 5 shows β-arrestin assay result (% bARR2 response) for compounds 4-9 on the δ-Opioid receptor (OPRD) vs a full agonist of OPRD, SNC80, and as compared to morphine.

FIGS. 6A-6C shows the results from a dominant assay for compounds 1, and stereoisomer compounds 4 and 5 using OPRM cell lines.

FIGS. 7A-7C shows the results from a dominant assay for racemic compound 1, and stereoisomer compounds 4 and 5 using OPRD cell lines.

FIGS. 8A-8C shows the results from a dominant assay for racemic compound 2, and stereoisomer compounds 6 and 7 using OPRM cell lines. All of compounds 2 (FIG. 8A), compound 6 (FIG. 8B), and compound 7 (FIG. 8C) were resistant to decreasing spare receptors.

FIGS. 9A-9C shows the results from a dominant assay for racemic compound 2, and stereoisomer compounds 6 and 7 using OPRD cell lines. All of compounds 2 (FIG. 9A), compound 6 (FIG. 9B), and compound 7 (FIG. 9C) were resistant to decreasing spare receptors.

FIGS. 10A-10C shows the results from a dominant assay for racemic compound 3, and stereoisomer compounds 8 and 9 using OPRM cell lines.

FIGS. 11A-11C shows the results from a dominant assay for racemic compound 3, and stereoisomer compounds 8 and 9 using OPRD cell lines.

### 5. DETAILED DESCRIPTION OF THE INVENTION

### 5.1 Opioid Receptor Agonist Compositions

As summarized above, the present disclosure provides compounds identified by the inventors as having dual agonist activity at μ and δ opioid receptors. The compounds of this disclosure can act as biased agonists of opioid receptors to provide GPCR functional selectivity, including selective activity in the Gi pathway. A “biased” agonist ligand of a G protein-coupled receptor (GPCR) is a compound that provides analgesic activity with reduced undesirable on-target side effects. The biased agonist compounds of this disclosure can exhibit reduced CNS and GI-related side effects as compared to opioid receptor agonists in general. In some embodiments, the opioid receptor agonist compounds provide for minimization or reduced levels of opioid receptor hyperalgesia.

More specifically, in an aspect, the present disclosure provides a pharmaceutical composition comprising:

(i) a compound of formula (I):

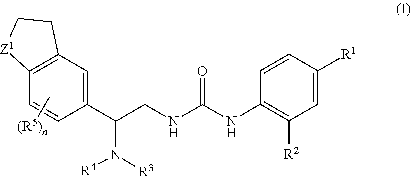

or a stereoisomer thereof, or a pharmaceutically acceptable salt thereof,
wherein:

Z¹ is selected from —O— and —NR⁶—, wherein R⁶ is selected from —H and optionally substituted (C₁-C₃)alkyl;

R¹, R² and each R⁵ are independently selected from H, halogen, OH, (C₁-C₃)alkyl, substituted (C₁-C₃)alkyl, (C₁-C₃)alkoxy, substituted (C₁-C₃)alkoxy; and

R³ and R⁴ are independently selected from —H and optionally substituted (C₁-C₃)alkyl; and

n is 0 to 3; and

(ii) a pharmaceutically acceptable excipient.

In some embodiments of formula (I), Z¹ is —NR⁶—. In some embodiments, R⁶ is (C₁-C₃)alkyl. In some embodiments of formula (I), Z¹ is —NCH₃—. In another embodiment of formula (I), R¹ is selected from —H and halogen, and R² is selected from —H and optionally substituted (C₁-C₃)alkyl. In some embodiments of formula (I), R¹ is —H. In some embodiments of formula (I), R¹ is halogen. In some embodiments of formula (I), R¹ is fluoro.

In some embodiments of formula (I), R² is —H. In some embodiments of formula (I), R² is optionally substituted (C₁-C₃)alkyl. In some embodiments of formula (I), R² is (C₁-C₃)alkyl, such as methyl.

In some embodiments of formula (I), Z¹ is —NR⁶—, R¹ is halogen and R² is —H. In some embodiments of formula (I), Z¹ is —NR⁶—, R¹ is fluoro and R² is —H. In another embodiment of formula (I), R³ and R⁴ are each (C₁-C₃)alkyl. In another embodiment of formula (I), R³ and R⁴ are each —CH₃. In another embodiment of formula (I), R³ is (C₁-C₃)alkyl and R⁴ is —H. In another embodiment of formula (I), R³ and R⁴ are each —H. In some embodiments of formula (I), n is 0 and no R⁵ substituent is present.

In some embodiments of the pharmaceutical composition, the compound of formula (I) is the compound:

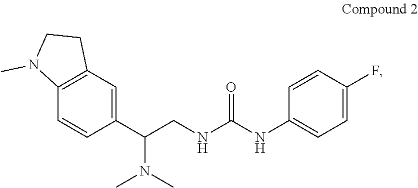 [SMILES: CN1CCc2cc(C(CNC(=O)Nc3ccc(F)cc3)N(C)C)ccc21]

or a substantially optically pure stereoisomer thereof, or a pharmaceutically acceptable salt thereof.

In some embodiments of formula (I), Z¹ is —O—. In another embodiment of formula (I), R¹ is selected from —H and halogen, and R² is selected from —H and optionally substituted (C₁-C₃)alkyl.

In some embodiments of formula (I), Z¹ is —O—, R¹ is —H and R² is an optionally substituted (C₁-C₃)alkyl. In some embodiments of formula (I), Z¹ is —O—, R¹ is —H and R² is methyl. In another embodiment of formula (I), R³ and R⁴ are each (C₁-C₃)alkyl. In another embodiment of formula (I), R³ and R⁴ are each —CH₃. In another embodiment of formula (I), R³ is (C₁-C₃)alkyl and R⁴ is —H. In another embodiment of formula (I), n is 0, no R⁵ substituent is present.

In some embodiments of the pharmaceutical composition, the compound of formula (I) is the compound:

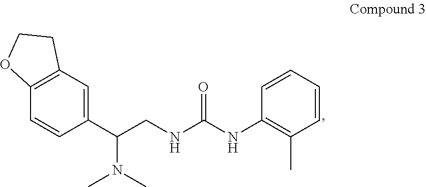 [SMILES: Cc1ccccc1NC(=O)NCC(c1ccc2c(c1)CCO2)N(C)C]

or a substantially optically pure stereoisomer thereof, or a pharmaceutically acceptable salt thereof.

In a second aspect, the present disclosure provides a pharmaceutical composition comprising:

(i) a compound of formula (II):

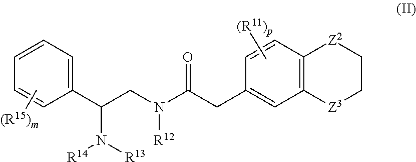

or a stereoisomer thereof, or a pharmaceutically acceptable salt thereof,
wherein:

Z² and Z³ are independently selected from —O— and —NR¹⁶—, wherein R¹⁶ is selected from —H and optionally substituted (C₁-C₃)alkyl; R¹², R¹³ and R¹⁴ are independently selected from —H and optionally substituted (C₁-C₃)alkyl; and

each R¹¹ and each R¹⁵ are independently selected from —H, -halogen, —OH, —CF₃, —OCF₃, —CN, —NH₂, —NO₂, optionally substituted (C₁-C₆)alkyl, optionally substituted (C₁-C₆)alkoxy, —C(O)OR¹², —C(O)NHR¹², —SO₂NHR¹², —NR¹³R¹⁴, —NHC(O)R¹², and —SO₃H;

m is 0 to 5;

p is 0 to 3; and

(ii) a pharmaceutically acceptable excipient.

In some embodiments of formula (II), R¹² is —H. In some embodiments of formula (II), R¹² is (C₁-C₃)alkyl, such as methyl.

In another embodiment of formula (II), R¹³ and R¹⁴ are each (C₁-C₃)alkyl. In another embodiment of formula (II), R¹³ and R¹⁴ are each —CH₃. In another embodiment of formula (II), R¹³ and R¹⁴ are each —H. In another embodiment of formula (II), R¹³ is (C₁-C₃)alkyl and R¹⁴ is —H.

In some embodiments of formula (II), Z² is —O—. In some embodiments of formula (II), Z³ is —O—. In some embodiments of formula (II), Z² is —NR¹⁶—. In some embodiments of formula (II), Z³ is —NR¹⁶—. In some embodiments, R¹⁶ is H. In some embodiments of formula (II), Z² and Z³ are each —O—.

In some embodiments of formula (II), m is 0. In some embodiments of formula (II), m is 1, 2, 3, 4 or 5. In some embodiments of formula (II), p is 0. In some embodiments of formula (II), p is 1, 2, or 3.

In some embodiments of formula (II), each R¹¹ and each R¹⁵ are independently selected from —H, -halogen (e.g., fluoro), —OH, optionally substituted (C₁-C₃)alkyl (e.g., —CF₃ or methyl), and optionally substituted (C₁-C₃)alkoxy (e.g., —OCF₃ or methoxy).

In some embodiments, the compound of formula (II) is a compound of formula (III):

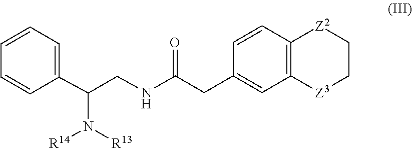

or a substantially optically pure stereoisomer thereof, or a pharmaceutically acceptable salt thereof.

In some embodiments of formula (III), Z² is —O—. In some embodiments of formula (III), Z³ is —O—. In some embodiments of formula (III), Z² is —NR¹⁶—. In some embodiments of formula (III), Z³ is —NR¹⁶—. In some embodiments, R¹⁶ is H. In some embodiments of formula (III), Z² and Z³ are each —O—.

In some embodiments of formula (III), R¹³ and R¹⁴ are each —H. In some embodiments of formula (III), R¹³ and R¹⁴ are each (C₁-C₃)alkyl. In some embodiments of formula (III), R¹³ and R¹⁴ are each —CH₃.

In some embodiments of the pharmaceutical composition, the compound of formula (III) is the compound:

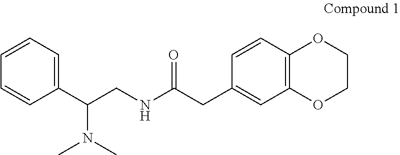 [SMILES: CN(C)C(CNC(=O)Cc1ccc2c(c1)OCCO2)c1ccccc1]

or a substantially optically pure stereoisomer thereof, or a pharmaceutically acceptable salt thereof.

In typical embodiments, the pharmaceutical compositions comprise a compound as described herein, a substantially optically pure compound as described herein, a pharmaceutically acceptable salt thereof, or a prodrug thereof, in a therapeutically effective amount.

In some embodiments, the pharmaceutical compositions are formulated for oral delivery.

In some embodiments, the pharmaceutical compositions are formulated for parenteral administration to a subject in need thereof. In some parenteral embodiments, the pharmaceutical compositions are formulated for intravenous administration to a subject in need thereof. In some parenteral embodiments, the pharmaceutical compositions are formulated for subcutaneous administration to a subject in need thereof.

### 5.1.1 Isotopically Labelled Analogs

The present disclosure also encompasses pharmaceutical compositions comprising isotopically-labeled compounds which are identical to those compounds as described herein, except that one or more atoms are replaced by an atom having an atomic mass or mass number different from the atomic mass or mass number usually found in nature (“isotopologues”). The compounds of the present disclosure may also contain unnatural proportions of atomic isotopes at one or more atoms that constituted such compounds.

Examples of isotopes that can be incorporated into compounds described herein include isotopes of hydrogen, carbon, nitrogen, oxygen, phosphorus, fluorine and chlorine, such as ²H (“D”), ³H, ¹³C, ¹⁴C, ¹⁵N, ¹⁸O, ¹⁷O, ³¹P, ³²p, ³⁵S, ¹⁸F, and ³⁶Cl, respectively. For example, a compound described herein can have one or more H atoms replaced with deuterium.

Unless otherwise stated, compounds described herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

In some embodiments of the pharmaceutical composition, certain isotopically-labeled compounds, such as those labeled with ³H and ¹⁴C, can be useful in compound and/or substrate tissue distribution assays. Tritiated (3H) and carbon-14 (¹⁴C) isotopes can be particularly preferred for their ease of preparation and detectability. Further, substitution with heavier isotopes such as deuterium can afford certain therapeutic advantages resulting from greater metabolic stability, such as increased in vivo half-life or reduced dosage requirements, and hence can be preferred in some circumstances. Isotopically-labeled compounds can generally be prepared by following procedures analogous to those disclosed herein, for example, in the Examples section, by substituting an isotopically-labeled reagent for a non-isotopically-labeled reagent.

In some embodiments, the compounds disclosed in the present disclosure are deuterated analogs of any of the compounds, or a salt thereof, as described herein. A deuterated analog of a compound of formulae (I)-(III) is a compound where one or more hydrogen atoms are substituted with a deuterium. In some embodiments, the deuterated analog is a compound of formulae (I)-(III) that includes a deuterated R¹, R², R³, R⁴, R⁵, R⁶, R¹¹, R¹², R¹³, R¹⁴, R¹⁵, or R¹⁶ group. In certain embodiments of a deuterated analog of a compound of formulae (I)-(III), R¹, R², R³, R⁴, R⁵, R⁶, R¹¹, R¹², R¹³, R¹⁴, R¹⁵, or R¹⁶ are independently selected from optionally substituted (C₁-C₃)alkyl, optionally substituted (C₁-C₃)alkoxy, optionally substituted (C₁-C₃)alkylene-heterocycloalkyl, optionally substituted monocyclic or bicyclic carbocycle, and optionally substituted monocyclic or bicyclic heterocycle including at least one deuterium atom. In certain embodiments of the deuterated analog of a compound of formulae (I)-(III), R, R², R³, R⁴, R⁵, R⁶, R¹¹, R¹², R¹³, R¹⁴, R¹, and R¹⁶ are independently selected from an optionally substituted (C₁-C₃) alkyl, an optionally substituted (C₁-C₃)alkoxy, and an optionally substituted (C₁-C₃)-alkylene-heterocycle (e.g. —(CH₂)ₘ-morpholine, —(CH₂)ₘ-piperazine, and —(CH₂)ₘ-piperidine). In some embodiments of a deuterated analog of a compound of formulae (I)-(III), the hydrogen atom of an aliphatic or an aromatic C—H bond is replaced by a fluorine atom.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

### 5.1.2 Fluorinated Analogs

In some embodiments, the compounds disclosed in the present disclosure are fluorinated analogs of any of the compounds, or a salt thereof, as described herein. A fluorinated analog of a compound of formulae (I)-(III) is a compound where one or more hydrogen atoms or substituents are substituted with a fluorine atom. In some embodiments, the fluorinated analog is a compound of formulae (I)-(III) that includes a R¹, R², R³, R⁴, R⁵, R⁶, R¹¹, R¹², R¹³, R¹⁴, R¹⁵, or R¹⁶ group. In some embodiments of a fluorinated analog of a compound of formulae (I)-(III), R¹, R², R³, R⁴, R⁵, R⁶, R¹¹, R¹², R¹³, R¹⁴, R⁵ and R¹⁶ groups are independently selected from optionally substituted (C₁-C₃)alkyl, optionally substituted (C₁-C₃)alkoxy, optionally substituted (C₁-C₃)alkylene-heterocycloalkyl, optionally substituted monocyclic or bicyclic carbocycle, optionally substituted monocyclic or bicyclic heterocycle, optionally substituted aryl, and optionally substituted heteroaryl including at least one fluorine atom. In some embodiments of a fluorinated analog of a compound of formulae (I)-(III), the hydrogen atom of an aliphatic or an aromatic C—H bond is replaced by a fluorine atom. In some embodiments of a fluorinated analog of a compound of formulae (I)-(III), at least one hydrogen of an optionally substituted aryl or an optionally substituted heteroaryl is replaced by a fluorine atom. In some embodiments of a fluorinated analog of a compound of formulae (I)-(III), a hydroxyl substituent (—OH) or an amino substituent (—NH₂) is replaced by a fluorine atom. In some embodiments of a fluorinated analog of a compound of formula (I), R¹, R², R³, R⁴, R⁵, R⁶, R, R¹², R¹³, R¹⁴, R¹⁵ and R¹⁶ groups are independently selected from F, CF₃, CF₂CF₃, CHF₂, OCF₃, OCHF₂, and OCF₂CF₃.

### 5.1.3 Salts, Polymorphs, Solvates, Hydrates and Other Forms

In some embodiments, the compounds described herein also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

In some embodiments, the compounds described herein are present in a solvate form.

In some embodiments, the compounds described herein are present in a hydrate form when the solvent component of the solvate is water.

In some embodiments, the compounds described herein have one or more chiral centers. It is understood that if an absolute stereochemistry is not expressly indicated, then all chiral, diastereomeric, and racemic forms of a compound are intended to be encompassed.

In some embodiments, the compounds described herein are present in a salt form.

In some embodiments, the compounds, or a prodrug form thereof, are provided in the form of pharmaceutically acceptable salts. Compounds containing an amine functional group or a nitrogen-containing heteroaryl group may be basic in nature and may react with any number of inorganic and organic acids to from the corresponding pharmaceutically acceptable salts. In some embodiments, the composition includes an acid addition salt form of the compound (e.g., as described herein). In some embodiments, the acid addition salt is an inorganic acid salt. In some embodiments, the acid addition salt is an organic acid salt.

It is understood that all variations of salts, solvates, hydrates, prodrugs and stereoisomers are meant to be encompassed by the present disclosure.

### 5.1.4 Prodrugs

Aspects of this disclosure include prodrug forms of any of the compounds described herein. Any convenient prodrug forms of the subject compounds can be prepared, for example, according to the strategies and methods described by Rautio et al. (“Prodrugs: design and clinical applications”, Nature Reviews Drug Discovery 7, 255-270 (February 2008)).

The term “prodrug” refers to an agent which is converted into a biologically active drug in vivo by some physiological or chemical process. In some embodiments, a prodrug is converted to the desired drug form, when subjected to a biological system at physiological pH. In some embodiments, a prodrug is enzymatically converted to the desired drug form, when subjected to a biological system.

Prodrugs forms of any of the compounds described herein can be useful, for example, to provide particular therapeutic benefits as a consequence of an extension of the half-life of the resulting compound in the body, or a reduction in the active dose required.

Pro-drugs can also be useful in some situations, as they may be easier to administer than the parent drug. They may, for instance, be bioavailable by oral administration whereas the parent drug is not. The pro-drug may also have improved solubility in pharmacological compositions over the parent drug.

Prodrug forms or derivatives of a compound of this disclosure generally include a promoiety substituent at a suitable labile site of the compound. The promoiety refers to the group that can be removed by enzymatic or chemical reactions, when a prodrug is converted to the drug in vivo.

In some embodiments, the promoiety is a group (e.g., a optionally substituted C₁₋₆ alkanoyl, or an optionally substituted C₁₋₆ alkyl) attached via an ester linkage to a hydroxyl group or a carboxylic acid group of the compound or drug.

### 5.1.5 Compound Synthesis

Compounds of the present disclosure may be synthesized according to standard methods known in the art. Some compounds and/or intermediates of the present disclosure may be commercially available, known in the literature, or readily obtainable by those skilled in the art using standard procedures. Some compounds of the present disclosure may be synthesized using schemes, examples, or intermediates described herein. Where the synthesis of a compound, intermediate or variant thereof is not fully described, those skilled in the art can recognize that the reaction time, number of equivalents of reagents and/or temperature may be modified from reactions described herein to prepare compounds presented or intermediates or variants thereof and that different work-up and/or purification techniques may be necessary or desirable to prepare such compounds, intermediates, or variants.

For example, compounds of formula (I) that include a central urea bond, e.g., compounds 2 and 3, may be prepared by a variety of methods, such as via a coupling between an amine fragment and a isocyanate fragment under suitable conditions. Such fragments or synthetic precursors thereof are readily available commercially. Protecting groups can be utilized on the fragments during the coupling reaction to avoid unwanted side reactions at e.g., a primary or secondary amino group that is present in one or both of the two fragments.

For example, compounds of formula (II) or (III) that include a central amide bond, e.g., compound 1, may be prepared by a coupling reaction between an amine fragment and a carboxylic acid fragment via activation of the carboxylic acid. Such fragments or synthetic precursors thereof are readily available commercially. Protecting groups can be utilized on the fragments during the coupling reaction to avoid unwanted side reactions at e.g., a primary or secondary amino group that is present in one or both of the two fragments.

Synthetic reactions may be carried out under various temperatures and/or atmospheric conditions to achieve desired results.

Synthetic reactions may be carried out in various reaction mixtures. Reaction mixtures may include water or other solvents. Such solvents may include organic or hydrophobic solvents. Reaction mixtures may be formulated with various compounds to alter one or more of pH and salinity. In some embodiments, reaction mixtures may include one or more reaction compounds. Reaction compounds may include reactants, catalysts, and/or other chemicals necessary for facilitating chemical reactions.

Filtration, concentration, and/or purification of compounds presented herein (or intermediates or variants thereof) may be carried out according to methods known in the art. Examples of purification methods may include chromatography, e.g., column chromatography. Chromatography may include, but is not limited to, one or more of thin-layer chromatography (TLC), preparative TLC (prep-TLC), normal phase chromatography, silica gel chromatography, flash silica gel chromatography, high performance liquid chromatography (HPLC), preparative HPLC (prep-HPLC), reverse phase column chromatography, reverse phase flash chromatography, C18 reverse phase flash chromatography, and C18 reverse phase HPLC. Filtration may be carried out, in some embodiments, over celite. Removal of water may be carried out, in some embodiments, using a Dean-Stack apparatus. In some embodiments, solids may be extracted from solution by lyophilization. In some embodiments, preparations may be sonicated before subsequent reactions and/or purification. In some embodiments, filtration and/or concentration may be carried out under varying pressure to achieve desired results. In some cases, filtration, concentration, and/or purification may be carried out in a vacuum. Compound preparations resulting from filtration, concentration, and/or purification may be in liquid or solid form. Liquids preparations may include water or other solvents. Such solvents may include organic solvents or hydrophobic solvents. Some compound preparations may be in the form of an oil. Solid compound preparations may include different formats that include, but are not limited to blocks, crystalline or granular formats, or powders. Filtration, concentration, and/or purification may be carried out using an eluant. Eluants may include water or other solvents. Such solvents may include organic or hydrophobic solvents. Some eluants may include ethyl acetate, petroleum ether, hexane, or n-hexane.

Synthesized compounds may be validated for proper structure by methods known to those skilled in the art, for example by nuclear magnetic resonance (NMR) spectroscopy and/or mass spectrometry.

### 5.2 Opioid Receptor Agonist Stereoisomer Compounds

As summarized above, the present disclosure provides substantially optically pure stereoisomers of compounds of this disclosure that are identified by the inventors as having dual agonist activity at μ and δ opioid receptors. In some embodiments, the substantially optically pure stereoisomer compounds have superior dual agonist activity as compared to the corresponding racemic parent compound.

### 5.2.1 Substantially Optically Pure (+) Stereoisomers of Formula (I)

In some embodiments, the (+) stereoisomer has superior dual agonist activity as compared to the racemic parent compound of this disclosure. In one embodiment, the present disclosure provides a substantially optically pure (+) stereoisomer of a compound of formula (I):

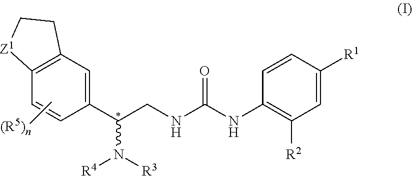

or a pharmaceutically acceptable salt thereof,
wherein:

Z¹ is selected from —O— and —NR⁶—, wherein R⁶ is selected from —H and optionally substituted (C₁-C₃)alkyl;

R¹, R² and each R⁵ are independently selected from H, halogen, OH, (C₁-C₃)alkyl, substituted (C₁-C₃)alkyl, (C₁-C₃)alkoxy, substituted (C₁-C₃)alkoxy; and

R³ and R⁴ are independently selected from —H and optionally substituted (C₁-C₃)alkyl; and

n is 0 to 3.

In some embodiments of the substantially optically pure (+) stereoisomer of a compound of formula (I), Z¹ is —NR⁶—. In some embodiments, R⁶ is (C₁-C₃)alkyl. In certain cases, —R⁶ is H. In certain cases, R⁶ is methyl or ethyl. In certain cases, R⁶ is propyl. In some embodiments, Z¹ is —NCH₃—. In another embodiment of a substantially optically pure (+) stereoisomer of the compound of formula (I), R¹ is selected from —H and halogen, and R² is selected from —H and optionally substituted (C₁-C₃)alkyl. In some embodiments, R¹ is —H.

In some embodiments, R¹ is halogen. In some embodiments, R¹ is fluoro. In some embodiments of the substantially optically pure (+) stereoisomer of a compound of formula (I), R² is —H. In some embodiments, R² is optionally substituted (C₁-C₃)alkyl. In some embodiments, R² is (C₁-C₃)alkyl, such as methyl.

In some embodiments of the substantially optically pure (+) stereoisomer of a compound of formula (I), Z¹ is —NR⁶—, R¹ is halogen and R² is —H. In some embodiments of the substantially optically pure (+) stereoisomer of a compound of formula (I), Z¹ is —NR⁶—, R¹ is fluoro and R² is —H.

In some embodiments of the substantially optically pure (+) stereoisomer of a compound of formula (I), n is 0 and no R⁵ substituent is present.

In some embodiments, the present disclosure provides a substantially optically pure (+) stereoisomer of a compound of the following structure:

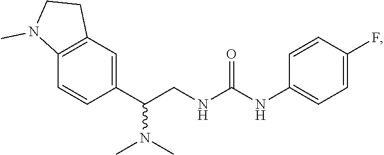 [SMILES: CN1CCc2cc(C(CNC(=O)Nc3ccc(F)cc3)N(C)C)ccc21]

or a pharmaceutically acceptable salt thereof.

In some embodiments of the substantially optically pure (+) stereoisomer of a compound of formula (I), Z¹ is —O—, R¹ is —H and R² is an optionally substituted (C₁-C₃)alkyl.

In some embodiments of the substantially optically pure (+) stereoisomer of a compound of formula (I), Z¹ is —O—, R¹ is —H and R² is methyl. In some embodiments, R³ and R⁴ are each (C₁-C₃)alkyl. In some embodiments of the substantially optically pure (+) stereoisomer of a compound of formula (I), R³ and R⁴ are each —CH₃. In some embodiments, R³ is (C₁-C₃)alkyl and R⁴ is —H.

In some embodiments of a substantially optically pure (+) stereoisomer of the compound of formula (I), n is 0, no R⁵ substituent is present.

In some embodiments, the present disclosure provides a substantially optically pure (+) stereoisomer of a compound of the following structure:

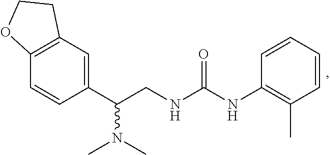 [SMILES: Cc1ccccc1NC(=O)NCC(c1ccc2c(c1)CCO2)N(C)C]

or a pharmaceutically acceptable salt thereof.

In some embodiments, the substantially optically pure (+) stereoisomer of a compound of formula (I), has an optical purity of 80% or more, such as 85% or more, 90% or more, 95% or more, 96% or more, 97% or more, 97.5% or more, 98% or more, 98.5% or more, 99% or more, or 99.5% or more. In some embodiments, the substantially optically pure (+) stereoisomer of a compound of formula (I), has an enantiomeric excess of 80% or more, such as an enantiomeric excess of 85% or more, 90% ee or more, 95% ee or more, 97.5% ee or more, 98% ee or more, or 99% ee or more.

### 5.2.2 Substantially Optically Pure (−) Stereoisomers of Formula (I)

In some embodiments, the (−) stereoisomer has superior dual agonist activity as compared to the racemic parent compound of this disclosure. In one embodiment, the present disclosure provides a substantially optically pure (−) stereoisomer of a compound of formula (I):

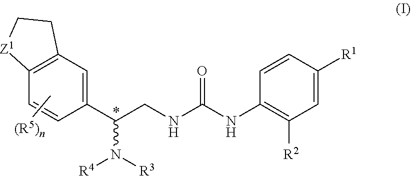

or a pharmaceutically acceptable salt thereof,
wherein:

Z¹ is selected from —O— and —NR⁶—, wherein R⁶ is selected from —H and optionally substituted (C₁-C₃)alkyl;

R¹, R² and each R⁵ are independently selected from H, halogen, OH, (C₁-C₃)alkyl, substituted (C₁-C₃)alkyl, (C₁-C₃)alkoxy, substituted (C₁-C₃)alkoxy; and

R³ and R⁴ are independently selected from —H and optionally substituted (C₁-C₃)alkyl; and

n is 0 to 3.

In some embodiments of the substantially optically pure (−) stereoisomer of a compound of formula (I), Z¹ is —NR⁶—. In some embodiments, R⁶ is (C₁-C₃)alkyl. In certain cases, —R⁶ is H. In certain cases, R⁶ is methyl or ethyl. In certain cases, R⁶ is propyl. In some embodiments, Z¹ is —NCH₃—. In some embodiments of the substantially optically pure (−) stereoisomer of a compound of formula (I), R¹ is selected from —H and halogen, and R² is selected from —H and optionally substituted (C₁-C₃)alkyl. In some embodiments, R¹ is —H. In some embodiments, R¹ is halogen. In some embodiments, R¹ is fluoro. In some embodiments of the substantially optically pure (−) stereoisomer of a compound of formula (I), R² is —H. In some embodiments, R² is optionally substituted (C₁-C₃)alkyl. In some embodiments, R² is (C₁-C₃)alkyl, such as methyl.

In some embodiments of the substantially optically pure (−) stereoisomer of a compound of formula (I), Z¹ is —NR⁶—, R¹ is halogen and R² is —H. In some embodiments of the substantially optically pure (−) stereoisomer of a compound of formula (I), Z¹ is —NR⁶—, R¹ is fluoro and R² is —H.

In some embodiments of the substantially optically pure (−) stereoisomer of the compound of formula (I), R³ and R⁴ are each (C₁-C₃)alkyl. In some embodiments, R³ and R⁴ are each —CH₃. In another embodiment of the substantially optically pure (−) stereoisomer of a compound of formula (I), R³ is (C₁-C₃)alkyl and R⁴ is —H. In some embodiments of formula (I), R³ and R⁴ are each —H.

In some embodiments of the substantially optically pure (−) stereoisomer of a compound of formula (I), n is 0 and no R⁵ substituent is present.

In some embodiments, the present disclosure provides a substantially optically pure (−) stereoisomer of a compound of the following structure:

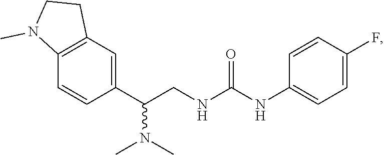 [SMILES: CN1CCc2cc(C(CNC(=O)Nc3ccc(F)cc3)N(C)C)ccc21]

or a pharmaceutically acceptable salt thereof.

In some embodiments of the substantially optically pure (−) stereoisomer of a compound of formula (I), Z¹ is —O—, R¹ is —H and R² is an optionally substituted (C₁-C₃)alkyl.

In some embodiments of the substantially optically pure (−) stereoisomer of a compound of formula (I), Z¹ is —O—, R¹ is —H and R² is methyl. In some embodiments, R³ and R⁴ are each (C₁-C₃)alkyl. In some embodiments of the substantially optically pure (−) stereoisomer of a compound of formula (I), R³ and R⁴ are each —CH₃. In some embodiments, R³ is (C₁-C₃)alkyl and R⁴ is —H.

In some embodiments of the substantially optically pure (−) stereoisomer of a compound of formula (I), n is 0, no R⁵ substituent is present.

In some embodiments, the present disclosure provides a substantially optically pure (−) stereoisomer of a compound of the following structure:

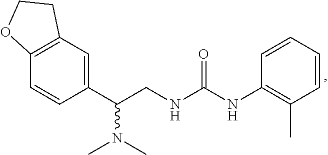 [SMILES: Cc1ccccc1NC(=O)NCC(c1ccc2c(c1)CCO2)N(C)C]

or a pharmaceutically acceptable salt thereof.

In some embodiments, the substantially optically pure (−) stereoisomer of a compound of formula (I), has an optical purity of 80% or more, such as 85% or more, 90% or more, 95% or more, 96% or more, 97% or more, 97.5% or more, 98% or more, 98.5% or more, 99% or more, or 99.5% or more. In some embodiments, the substantially optically pure (−) stereoisomer of a compound of formula (I), has an enantiomeric excess of 80% or more, such as an enantiomeric excess of 85% or more, 90% ee or more, 95% ee or more, 97.5% ee or more, 98% ee or more, or 99% ee or more.

### 5.2.3 Substantially Optically Pure (+) Stereoisomers of Formula (II)

In one embodiment, the present disclosure provides a substantially optically pure (+) stereoisomer of a compound of formula (II):

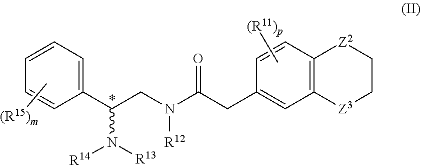

or a stereoisomer thereof, or a pharmaceutically acceptable salt thereof,
wherein:

Z² and Z³ are independently selected from —O— and —NR¹⁶—, wherein R¹⁶ is selected from —H and optionally substituted (C₁-C₃)alkyl;

R¹², R¹³ and R¹⁴ are independently selected from —H and optionally substituted (C₁-C₃)alkyl;

each R¹¹ and each R¹⁵ are independently selected from —H, -halogen, —OH, —CF₃, —OCF₃, —CN, —NH₂, —NO₂, optionally substituted (C₁-C₆)alkyl, optionally substituted (C₁-C₆)alkoxy, —C(O)OR¹², —C(O)NHR¹², —SO₂NHR¹², —NR¹³R¹⁴, —NHC(O)R¹², and —SO₃H;

m is 0 to 5; and

p is 0 to 3.

In some embodiments of the substantially optically pure (+) stereoisomer of a compound of formula (II), R¹² is —H. In some embodiments of formula (II), R¹² is (C₁-C₃)alkyl, such as methyl.

In some embodiment of the substantially optically pure (+) stereoisomer of a compound of formula (II), R¹³ and R¹⁴ are each (C₁-C₃)alkyl. In another embodiment, R¹³ and R¹⁴ are each —CH₃. In another embodiment, R¹³ and R¹⁴ are each —H. In another embodiment, R¹³ is (C₁-C₃)alkyl and R¹⁴ is —H.

In some embodiments of the substantially optically pure (+) stereoisomer of a compound of formula (II), Z² is —O—. In some embodiments of the substantially optically pure (+) stereoisomer of a compound of formula (II), Z³ is —O—. In some embodiments of the substantially optically pure (+) stereoisomer of a compound of formula (II), Z² is —NR¹⁶—. In some embodiments of the substantially optically pure (+) stereoisomer of a compound of formula (II), Z³ is —NR¹⁶—. In some embodiments, R¹⁶ is H. In some embodiments, Z² and Z³ are each —O—.

In some embodiments of the substantially optically pure (+) stereoisomer of a compound of formula (II), m is 0. In some embodiments, m is 1, 2, 3, 4 or 5. In some embodiments of the substantially optically pure (+) stereoisomer of a compound of formula (II), p is 0. In some embodiments, p is 1, 2, or 3.

In some embodiments of a substantially optically pure (+) stereoisomer of a compound of formula (II), each R¹¹ and each R¹⁵ are independently selected from —H, -halogen (e.g., fluoro), —OH, optionally substituted (C₁-C₃)alkyl (e.g., —CF₃ or methyl), and optionally substituted (C₁-C₃)alkoxy (e.g., —OCF₃ or methoxy).

In some embodiments, the substantially optically pure (+) stereoisomer of a compound of formula (II) is a compound of formula (III):

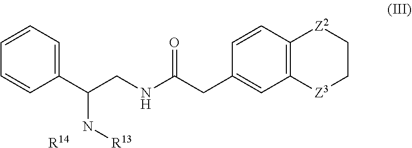

or a stereoisomer thereof, or a pharmaceutically acceptable salt thereof.

In some embodiments of the substantially optically pure (+) stereoisomer of a compound of formula (III), Z² is —O—. In some embodiments of the substantially optically pure (+) stereoisomer of a compound of formula (III), Z³ is —O—. In some embodiments of a substantially optically pure (+) stereoisomer of a compound of formula (III), Z² is —NR¹⁶—. In some embodiments of the substantially optically pure (+) stereoisomer of a compound of formula (III), Z³ is —NR¹⁶—. In some embodiments, R¹⁶ is H. In some embodiments, Z² and Z³ are each —O—.

In some embodiments of the substantially optically pure (+) stereoisomer of a compound of formula (II) or (III), R¹³ and R¹⁴ are each —H. In some embodiments of the substantially optically pure (+) stereoisomer of a compound of formula (II) or (III), R¹³ and R¹⁴ are each (C₁-C₃)alkyl. In some embodiments, R¹³ and R¹⁴ are each —CH₃.

In some embodiments, the present disclosure provides a substantially optically pure (+) stereoisomer of a compound of the following structure:

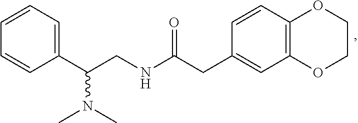 [SMILES: CN(C)C(CNC(=O)Cc1ccc2c(c1)OCCO2)c1ccccc1]

or a pharmaceutically acceptable salt thereof.

In some embodiments, the substantially optically pure (+) stereoisomer of a compound of formula (II)-(III), has an optical purity of 80% or more, such as 85% or more, 90% or more, 95% or more, 96% or more, 97% or more, 97.5% or more, 98% or more, 98.5% or more, 99% or more, or 99.5% or more. In some embodiments, the substantially optically pure (+) stereoisomer of a compound of formula (II)-(III), has an enantiomeric excess of 80% or more, such as an enantiomeric excess of 85% or more, 90% ee or more, 95% ee or more, 97.5% ee or more, 98% ee or more, or 99% ee or more.

### 5.2.4 Substantially Optically Pure (−) Stereoisomers of Formula (II)

In one embodiment, the present disclosure provides a substantially optically pure (−) stereoisomer of a compound of formula (II):

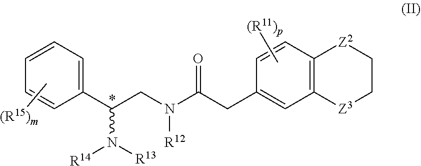

or a stereoisomer thereof, or a pharmaceutically acceptable salt thereof,
wherein:

Z² and Z³ are independently selected from —O— and —NR¹⁶—, wherein R¹⁶ is selected from —H and optionally substituted (C₁-C₃)alkyl;

R¹², R¹³ and R¹⁴ are independently selected from —H and optionally substituted (C₁-C₃)alkyl;

each R¹¹ and each R¹⁵ are independently selected from —H, -halogen, —OH, —CF₃, —OCF₃, —CN, —NH₂, —NO₂, optionally substituted (C₁-C₆)alkyl, optionally substituted (C₁-C₆)alkoxy, —C(O)OR¹², —C(O)NHR¹², —SO₂NHR¹², —NR¹³R¹⁴, —NHC(O)R¹², and —SO₃H;

m is 0 to 5; and

p is 0 to 3.

In some embodiments of the substantially optically pure (−) stereoisomer of a compound of formula (II), R¹² is —H. In some embodiments, R¹² is (C₁-C₃)alkyl, such as methyl.

In some embodiment of the substantially optically pure (−) stereoisomer of a compound of formula (II), R¹³ and R¹⁴ are each (C₁-C₃)alkyl. In another embodiment, R¹³ and R¹⁴ are each —CH₃. In another embodiment, R¹³ and R¹⁴ are each —H. In another embodiment, R¹³ is (C₁-C₃)alkyl and R¹⁴ is —H.

In some embodiments of the substantially optically pure (−) stereoisomer of a compound of formula (II), Z² is —O—. In some embodiments of the substantially optically pure (−) stereoisomer of a compound of formula (II), Z³ is —O—. In some embodiments of the substantially optically pure (−) stereoisomer of a compound of formula (II), Z² is —NR¹⁶—. In some embodiments of the substantially optically pure (−) stereoisomer of a compound of formula (II), Z³ is —NR¹⁶—. In some embodiments, R¹⁶ is H. In some embodiments, Z² and Z³ are each —O—.

In some embodiments of the substantially optically pure (−) stereoisomer of a compound of formula (II), m is 0. In some embodiments, m is 1, 2, 3, 4 or 5. In some embodiments of the substantially optically pure (−) stereoisomer of a compound of formula (II), p is 0. In some embodiments, p is 1, 2, or 3.

In some embodiments of a substantially optically pure (−) stereoisomer of a compound of formula (II), each R¹¹ and each R¹⁵ are independently selected from —H, -halogen (e.g., fluoro), —OH, optionally substituted (C₁-C₃)alkyl (e.g., —CF₃ or methyl), and optionally substituted (C₁-C₃)alkoxy (e.g., —OCF₃ or methoxy).

In some embodiments, the substantially optically pure (−) stereoisomer of a compound of formula (II) is a compound of formula (III):

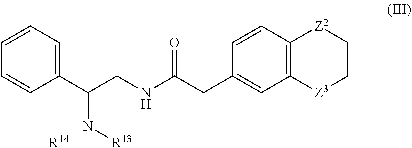

or a stereoisomer thereof, or a pharmaceutically acceptable salt thereof.

In some embodiments of the substantially optically pure (−) stereoisomer of a compound of formula (III), Z² is —O—. In some embodiments of the substantially optically pure (−) stereoisomer of a compound of formula (III), Z³ is —O—. In some embodiments of a substantially optically pure (−) stereoisomer of a compound of formula (III), Z² is —NR¹⁶—. In some embodiments of the substantially optically pure (−) stereoisomer of a compound of formula (III), Z³ is —NR¹⁶—. In some embodiments, R¹⁶ is H. In some embodiments, Z² and Z³ are each —O—.

In some embodiments of the substantially optically pure (−) stereoisomer of a compound of formula (II) or (III), R¹³ and R¹⁴ are each —H. In some embodiments of the substantially optically pure (−) stereoisomer of a compound of formula (II) or (III), R¹³ and R¹⁴ are each (C₁-C₃)alkyl. In some embodiments, R¹³ and R¹⁴ are each —CH₃.

In some embodiments, the present disclosure provides a substantially optically pure (−) stereoisomer of a compound of the following structure:

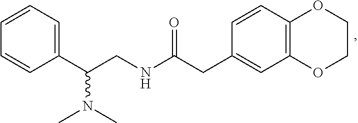 [SMILES: CN(C)C(CNC(=O)Cc1ccc2c(c1)OCCO2)c1ccccc1]

or a pharmaceutically acceptable salt thereof.

In some embodiments, the substantially optically pure (−) stereoisomer of a compound of formula (II)-(III), has an optical purity of 80% or more, such as 85% or more, 90% or more, 95% or more, 96% or more, 97% or more, 97.5% or more, 98% or more, 98.5% or more, 99% or more, or 99.5% or more. In some embodiments, the substantially optically pure (−) stereoisomer of a compound of formula (II)-(III), has an enantiomeric excess of 80% or more, such as an enantiomeric excess of 85% or more, 90% ee or more, 95% ee or more, 97.5% ee or more, 98% ee or more, or 99% ee or more.

### 5.3 Methods

### 5.2.5 Modulation of Opioid Receptor

In a third aspect, the present disclosure provides a method of modulating the activity of an opioid receptor, comprising contacting a biological system with a pharmaceutical composition as described herein. In some embodiments, the compound has agonist activity at the μ and/or δ opioid receptors. In some embodiments, the compound has dual agonist activity at the μ and δ opioid receptors.

In some embodiments, the compounds of the pharmaceutical composition are considered biased agonists of an opioid receptor. In another embodiment, the compounds of the pharmaceutical composition are biased agonist of the μ-opioid receptor. A biased μ-opioid receptor agonist is an agonist that does not significantly recruit β-arrestins, but can activate G-protein-dependent pathways in a biological system.

In some embodiments, the agonists compounds described herein selectively activates the G-protein pathway.

In some embodiments of the method of modulating the activity of an opioid receptor, the biological system is in vitro.

In some embodiments of the method of modulating the activity of an opioid receptor, the biological system is in vivo.

In some embodiments of the method of modulating the activity of an opioid receptor, the compound of the pharmaceutical composition is

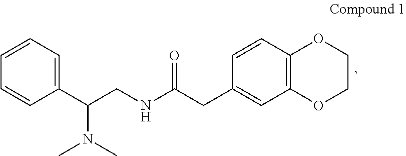 [SMILES: CN(C)C(CNC(=O)Cc1ccc2c(c1)OCCO2)c1ccccc1]

or a stereoisomer thereof, or a pharmaceutically acceptable salt thereof.

In some embodiments of the method of modulating the activity of an opioid receptor, the compound of the pharmaceutical composition is a substantially optically pure (+) stereoisomer of a compound of the following structure:

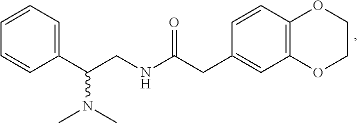 [SMILES: CN(C)C(CNC(=O)Cc1ccc2c(c1)OCCO2)c1ccccc1]

or a pharmaceutically acceptable salt thereof.

In some embodiments of the method of modulating the activity of an opioid receptor, the compound of the pharmaceutical composition is a substantially optically pure (−) stereoisomer of a compound of the following structure:

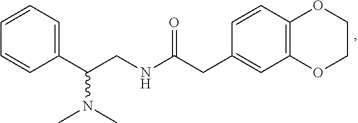 [SMILES: CN(C)C(CNC(=O)Cc1ccc2c(c1)OCCO2)c1ccccc1]

or a pharmaceutically acceptable salt thereof.

In some embodiments of the method of modulating the activity of an opioid receptor, the compound of the pharmaceutical composition is

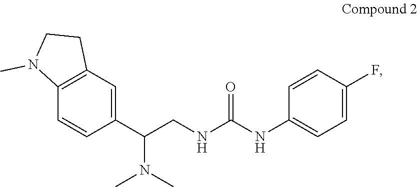 [SMILES: CN1CCc2cc(C(CNC(=O)Nc3ccc(F)cc3)N(C)C)ccc21]

or a stereoisomer thereof, or a pharmaceutically acceptable salt thereof.

In some embodiments of the method of modulating the activity of an opioid receptor, the compound of the pharmaceutical composition is a substantially optically pure (+) stereoisomer of a compound of the following structure:

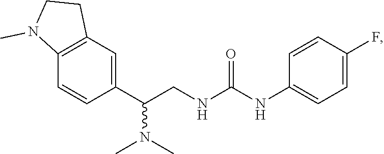 [SMILES: CN1CCc2cc(C(CNC(=O)Nc3ccc(F)cc3)N(C)C)ccc21]

or a pharmaceutically acceptable salt thereof.

In some embodiments of the method of modulating the activity of an opioid receptor, the compound of the pharmaceutical composition is a substantially optically pure (−) stereoisomer of a compound of the following structure:

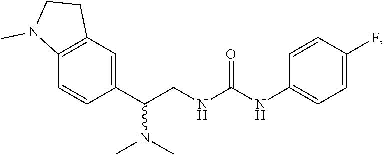 [SMILES: CN1CCc2cc(C(CNC(=O)Nc3ccc(F)cc3)N(C)C)ccc21]

or a pharmaceutically acceptable salt thereof.

In some embodiments of the method of modulating the activity of an opioid receptor, the compound of the pharmaceutical composition is

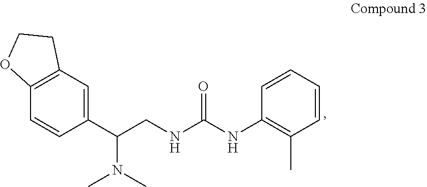 [SMILES: Cc1ccccc1NC(=O)NCC(c1ccc2c(c1)CCO2)N(C)C]

or a stereoisomer thereof, or a pharmaceutically acceptable salt thereof.

In some embodiments of the method of modulating the activity of an opioid receptor, the compound of the pharmaceutical composition is a substantially optically pure (+) stereoisomer of a compound of the following structure:

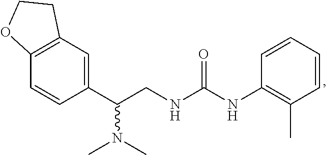 [SMILES: Cc1ccccc1NC(=O)NCC(c1ccc2c(c1)CCO2)N(C)C]

or a pharmaceutically acceptable salt thereof.

In some embodiments of the method of modulating the activity of an opioid receptor, the compound of the pharmaceutical composition is a substantially optically pure (−) stereoisomer of a compound of the following structure:

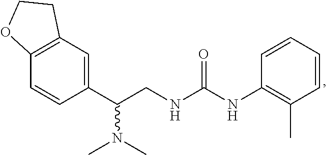 [SMILES: Cc1ccccc1NC(=O)NCC(c1ccc2c(c1)CCO2)N(C)C]

or a pharmaceutically acceptable salt thereof.

### 5.2.6 Method of Treating Pain

In a final aspect, the present disclosure provides a method of treating pain, comprising administering to a subject having pain an effective amount of a pharmaceutical composition as described herein to treat the subject for pain.

In some embodiments, the methods include administration of a biased μ-opioid and/or δ-opioid receptor agonist that does not significantly recruit β-arrestins, but can activate G-protein-dependent pathways, and thus can be administered without risk of significant undesirable side effects exhibited by conventional opioid receptor agonists, e.g., a μ-opioid agonist such as TRV130 (oliceridine). TRV130 is an opioid medication used for the treatment of moderate to severe acute pain in adults. It is given by intravenous (IV) injection, and can lead to side effects such as nausea, vomiting, dizziness, headache, constipation, itchy skin and low oxygen levels in blood.

In some embodiments of the method of treating pain, the compound of the pharmaceutical composition is:

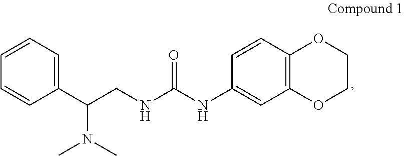 [SMILES: CN(C)C(CNC(=O)Nc1ccc2c(c1)OCCO2)c1ccccc1]

or a stereoisomer thereof, or a pharmaceutically acceptable salt thereof.

In some embodiments of the method of treating pain, the compound of the pharmaceutical composition is a substantially optically pure (+) stereoisomer of a compound of the following structure:

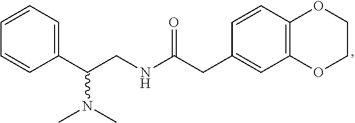 [SMILES: CN(C)C(CNC(=O)Cc1ccc2c(c1)OCCO2)c1ccccc1]

or a pharmaceutically acceptable salt thereof.

In some embodiments of the method of treating pain, the compound of the pharmaceutical composition is a substantially optically pure (−) stereoisomer of a compound of the following structure:

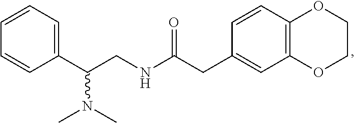 [SMILES: CN(C)C(CNC(=O)Cc1ccc2c(c1)OCCO2)c1ccccc1]

or a pharmaceutically acceptable salt thereof.

In some embodiments of the method of treating pain, the compound of the pharmaceutical composition is

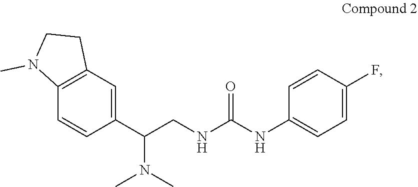 [SMILES: CN1CCc2cc(C(CNC(=O)Nc3ccc(F)cc3)N(C)C)ccc21]

or a stereoisomer thereof, or a pharmaceutically acceptable salt thereof.

In some embodiments of the method of treating pain, the compound of the pharmaceutical composition is a substantially optically pure (+) stereoisomer of a compound of the following structure:

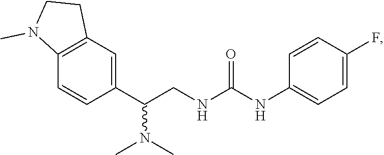 [SMILES: CN1CCc2cc(C(CNC(=O)Nc3ccc(F)cc3)N(C)C)ccc21]

or a pharmaceutically acceptable salt thereof.

In some embodiments of the method of treating pain, the compound of the pharmaceutical composition is a substantially optically pure (−) stereoisomer of a compound of the following structure:

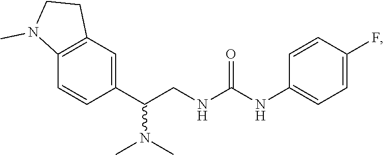 [SMILES: CN1CCc2cc(C(CNC(=O)Nc3ccc(F)cc3)N(C)C)ccc21]

or a pharmaceutically acceptable salt thereof.

In some embodiments of the method of treating pain, the compound of the pharmaceutical composition is

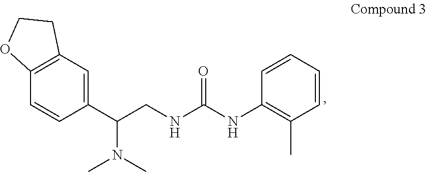 [SMILES: Cc1ccccc1NC(=O)NCC(c1ccc2c(c1)CCO2)N(C)C]

or a stereoisomer thereof, or a pharmaceutically acceptable salt thereof.

In some embodiments of the method of treating pain, the compound of the pharmaceutical composition is a substantially optically pure (+) stereoisomer of a compound of the following structure:

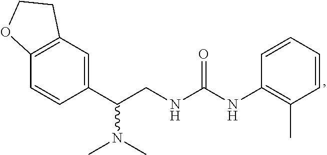 [SMILES: Cc1ccccc1NC(=O)NCC(c1ccc2c(c1)CCO2)N(C)C]

or a pharmaceutically acceptable salt thereof.

In some embodiments of the method of treating pain, the compound of the pharmaceutical composition is a substantially optically pure (−) stereoisomer of a compound of the following structure:

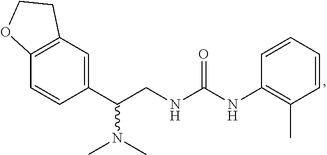 [SMILES: Cc1ccccc1NC(=O)NCC(c1ccc2c(c1)CCO2)N(C)C]

or a pharmaceutically acceptable salt thereof.

### 5.4 Definitions

Unless defined otherwise, all technical and scientific terms used herein have the same meaning as commonly understood by one of ordinary skill in the art to which this disclosure pertains.

It is understood that the definitions provided herein are not intended to be mutually exclusive. Accordingly, some chemical moieties may fall within the definition of more than one term.

As used herein, the symbol“ [symbol] ” refers to a covalent bond comprising a single or a double bond.

The term “Cₓ-C_{y}” when used in conjunction with a chemical moiety, such as alkyl, alkenyl, or alkynyl is meant to include groups that contain from x to y carbons in the chain. For example, the term “C₁-C₆ alkyl” refers to substituted or unsubstituted saturated hydrocarbon groups, including straight-chain alkyl and branched-chain alkyl groups that contain from 1 to 6 carbons. In some embodiments, the term “(Cₓ-C_{y})alkylene” refers to a substituted or unsubstituted alkylene chain with from x to y carbons in the alkylene chain. For example “(Cₓ-C_{y})alkylene may be selected from methylene, ethylene, propylene, butylene, pentylene, and hexylene, any one of which is optionally substituted.

The term “alkyl” refers to an unbranched or branched saturated hydrocarbon chain. In some embodiments, alkyl as used herein has 1 to 20 carbon atoms ((C₁-C₂₀)alkyl), 1 to 10 carbon atoms ((C₁-C₁₀)alkyl), 1 to 8 carbon atoms ((C₁-C₈)alkyl), 1 to 6 carbon atoms ((C₁-C₆)alkyl), or 1 to 5 carbon atoms ((C₁-C₅)alkyl). Examples include, but are not limited to, methyl, ethyl, n-propyl, isopropyl, n-butyl, sec-butyl, tert-butyl, n-pentyl, 2-pentyl, isopentyl, neopentyl, n-hexyl, 2-hexyl, 3-hexyl, and 3-methyl pentyl. When an alkyl residue having a specific number of carbons is named, all geometric isomers having that number of carbons may be encompassed. For example, “butyl” can include n-butyl, sec-butyl, isobutyl and t-butyl, and “propyl” can include n-propyl and isopropyl. Unless stated otherwise specifically in the specification, an alkyl chain is optionally substituted by one or more substituents such as those substituents described herein.

The term “alkylene” refers to a straight divalent hydrocarbon chain linking the rest of the molecule to a radical group, consisting solely of carbon and hydrogen, containing no unsaturation, and preferably having from 1 to 20 carbon atoms ((C₁-C₂₀)alkylene), 1 to 10 carbon atoms ((C₁-C₁₀)alkylene), 1 to 6 carbon atoms ((C₁-C₆)alkylene), or 1 to 5 carbon atoms ((C₁-C₅)alkylene). Examples include, but are not limited to, methylene, ethylene, propylene, butylene, and the like. The alkylene chain is attached to the rest of the molecule through a single bond and to the radical group through a single bond.

The points of attachment of the alkylene chain to the rest of the molecule and to the radical group are through the terminal carbons respectively. Unless stated otherwise specifically in the specification, an alkylene chain is optionally substituted by one or more substituents such as those substituents described herein. Examples include, methylene (—CH₂—), ethylene (—CH₂CH₂—), propylene (—CH₂CH₂CH₂—), 2-methylpropylene (—CH₂—CH(CH₃)—CH₂—), hexylene (—(CH₂)₆—) and the like.

The term “alkenyl” refers to an aliphatic hydrocarbon group containing at least one carbon-carbon double bond including straight-chain, branched-chain and cyclic alkenyl groups. In some embodiments, the alkenyl group has 2-10 carbon atoms (a C₂₋₁₀ alkenyl). In another embodiment, the alkenyl group has 2-4 carbon atoms in the chain (a C₂₋₄ alkenyl). Exemplary alkenyl groups include, but are not limited to, ethenyl, propenyl, n-butenyl, i-butenyl, 3-methylbut-2-enyl, n-pentenyl, heptenyl, octenyl, cyclohexyl-butenyl and decenyl. An alkylalkenyl is an alkyl group as defined herein bonded to an alkenyl group as defined herein. The alkenyl group can be unsubstituted or substituted through available carbon atoms with one or more groups defined hereinabove for alkyl

The term “alkynyl” refers to straight or branched monovalent hydrocarbyl groups having from 2 to 6 carbon atoms and preferably 2 to 3 carbon atoms and having at least 1 and preferably from 1 to 2 sites of acetylenic (C≡C—) unsaturation. Examples of such alkynyl groups include, but are not limited to, acetylenyl (C≡CH), and propargyl (CH₂C≡CH).

The term “aryl” refers to a monocyclic or polycyclic group having at least one hydrocarbon aromatic ring, wherein all of the ring atoms of the at least one hydrocarbon aromatic ring is carbon. Aryl may include groups with a single aromatic ring (e.g., phenyl) and multiple fused aromatic rings (e.g., naphthyl, anthryl). Aryl may further include groups with one or more aromatic hydrocarbon rings fused to one or more non-aromatic hydrocarbon rings (e.g., fluorenyl; 2,3-dihydro-1H-indene; 1,2,3,4-tetrahydronaphthalene). In certain embodiments, aryl includes groups with an aromatic hydrocarbon ring fused to a non-aromatic ring, wherein the non-aromatic ring comprises at least one ring heteroatom independently selected from the group consisting of N, O, and S. For example, in some embodiments, aryl includes groups with a phenyl ring fused to a non-aromatic ring, wherein the non-aromatic ring comprises at least one ring heteroatom independently selected from the group consisting of N, O, and S (e.g., chromane; thiochromane; 2,3-dihydrobenzofuran; indoline). In some embodiments, aryl as used herein has from 6 to 14 carbon atoms ((C₆-C₁₄)aryl), or 6 to 10 carbon atoms ((C₆-C₁₀)aryl). Where the aryl includes fused rings, the aryl may connect to one or more substituents or moieties of the formulae described herein through any atom of the fused ring for which valency permits.

The term “cycloalkyl” refers to a monocyclic or polycyclic saturated hydrocarbon. In some embodiments, cycloalkyl has 3 to 20 carbon atoms ((C₃-C₂₀)cycloalkyl), 3 to 8 carbon atoms ((C₃-C₅)cycloalkyl), 3 to 6 carbon atoms ((C₃-C₆)cycloalkyl), or 3 to 5 carbon atoms ((C₃-C₅)cycloalkyl). In some embodiments, cycloalkyl has 3 to 8 carbon atoms having single or multiple cyclic rings including fused, bridged, and spiro ring systems. Examples of suitable cycloalkyl groups include, but are not limited to, adamantyl, cyclopropyl, cyclobutyl, cyclopentyl, cyclooctyl, octahydropentalenyl, octahydro-1H-indene, decahydronaphthalene, cubane, bicyclo[3.1.0]hexane, and bicyclo[1.1.1]pentane, and the like.

The term “carbocycle” refers to a saturated, unsaturated or aromatic ring in which each atom of the ring is carbon. Carbocycle includes 3- to 10-membered monocyclic rings, 6- to 12-membered bicyclic rings, and 6- to 12-membered bridged rings. Each ring of a bicyclic carbocycle may be selected from saturated, unsaturated, and aromatic rings. In an exemplary embodiment, an aromatic ring, e.g., phenyl, may be fused to a saturated or unsaturated ring, e.g., cyclohexane, cyclopentane, or cyclohexene. A bicyclic carbocycle includes any combination of saturated, unsaturated and aromatic bicyclic rings, as valence permits. A bicyclic carbocycle includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. Exemplary carbocycles include cyclopentyl, cyclohexyl, cyclohexenyl, adamantyl, phenyl, indanyl, and naphthyl.

The term “heterocycle” refers to a saturated, unsaturated or aromatic ring comprising one or more heteroatoms. Exemplary heteroatoms include N, O, Si, P, B, and S atoms. Heterocycles include 3- to 10-membered monocyclic rings, 6- to 12-membered bicyclic rings, and 6- to 12-membered bridged rings. A bicyclic heterocycle includes any combination of saturated, unsaturated and aromatic bicyclic rings, as valence permits. In an exemplary embodiment, an aromatic ring, e.g., pyridyl, may be fused to a saturated or unsaturated ring, e.g., cyclohexane, cyclopentane, morpholine, piperidine or cyclohexene. A bicyclic heterocycle includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems.

The term “heteroaryl” refers to an aromatic group of from 4 to 10 carbon atoms and 1 to 4 heteroatoms selected from the group consisting of oxygen, nitrogen and sulfur within the ring. Such heteroaryl groups can have a single ring (i.e., pyridinyl or furyl) or multiple condensed rings (i.e., indolizinyl or benzothienyl) wherein the condensed rings may or may not be aromatic and/or contain a heteroatom provided that the point of attachment is through an atom of the aromatic heteroaryl group. In one embodiment, the nitrogen and/or the sulfur ring atom(s) of the heteroaryl group are optionally oxidized to provide for the N oxide (N→O), sulfinyl, or sulfonyl moieties. Preferred heteroaryls include 5 or 6 membered heteroaryls such as pyridinyl, pyrrolyl, indolyl, thiophenyl, and furanyl.

The term “heteroalkyl” refers to an alkyl substituent in which one or more of the carbon atoms and any attached hydrogen atoms are independently replaced with the same or different heteroatomic group. For example, 1, 2, or 3 carbon atoms may be independently replaced with the same or different heteroatomic substituent.

The term “substituted” refers to moieties having substituents replacing a hydrogen on one or more carbons or substitutable heteroatoms, e.g., an NH or NH₂ of a compound. It will be understood that “substitution” or “substituted with” includes the implicit proviso that such substitution is in accordance with permitted valence of the substituted atom and the substituent, and that the substitution results in a stable compound. For example, stable compounds include, but is not limited to, compounds which do not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds.

It will be understood by those skilled in the art that substituents can themselves be substituted, if appropriate. Unless specifically stated as “unsubstituted,” references to chemical moieties herein are understood to include substituted variants. For example, reference to a “heteroaryl” group or moiety implicitly includes both substituted and unsubstituted variants, unless specified otherwise.

The phrase “optionally substituted” refers to when a non-hydrogen substituent may or may not be present on a given atom, and, thus, the description includes structures wherein a non-hydrogen substituent is present and structures wherein a non-hydrogen substituent is not present.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2). In another exemplary embodiment, substituents include alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl, any of which may be optionally substituted by alkyl, alkenyl, alkynyl, halogen, haloalkyl, haloalkenyl, haloalkynyl, oxo, thioxo, cyano, nitro, imino, oximo, hydrazine, —R^{b}OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{a}, R^{b}, and R^{c} are independently selected from hydrogen, alkyl, cycloalkyl, cycloalkylalkyl, aryl, aralkyl, heterocycloalkyl, heterocycloalkylalkyl, heteroaryl, and heteroarylalkyl; and wherein each R^{a}, R^{b}, and R^{c}, valence permitting, may be optionally substituted with alkyl, alkenyl, alkynyl, halogen, haloalkyl, haloalkenyl, haloalkynyl, oxo, thioxo, cyano, nitro, imino, oximo, hydrazine, —R^{b}OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2).

The term “isomers” refers to two or more compounds comprising the same numbers and types of atoms, groups or components, but with different structural arrangement and connectivity of the atoms.

The term “tautomer” refers to one of two or more structural isomers which readily convert from one isomeric form to another and which exist in equilibrium.

A “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are non-superimposeable mirror images of one another.

The term “substantially optically pure” as used herein when referring to a particular compound means that, in any given sample of that compound, at least 80% by weight of one stereoisomer of the compound and less than about 20% by weight of other stereoisomers of the compound, more preferably greater than about 90% by weight of one stereoisomer of the compound and less than about 10% by weight of the other stereoisomers of the compound, more preferably greater than about 95% by weight of one stereoisomer of the compound and less than about 5% by weight of the other stereoisomers of the compound, more preferably greater than about 97% by weight of one stereoisomer of the compound and less than about 3% by weight of the other stereoisomers of the compound, more preferably greater than about 98% by weight of one stereoisomer of the compound and less than about 2% by weight of the other stereoisomers of the compound, and most preferably greater than about 99% by weight of one stereoisomer of the compound and less than about 1% by weight of the other stereoisomers of the compound. The optical purity can be determined by high performance liquid phase chromatography (HPLC) on a chiral stationary phase.

“Enantiomeric excess” (ee) of an enantiomer, when expressed as a percentage, is [(the mole fraction of the major enantiomer) minus (the mole fraction of the minor enantiomer)]×100.

Individual enantiomers and diastereomers of compounds of the present disclosure can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, (3) direct separation of the mixture of optical enantiomers on chiral liquid chromatographic columns, or (4) kinetic resolution using stereoselective chemical or enzymatic reagents. Racemic mixtures also can be resolved into their respective enantiomers by well-known methods, such as chiral-phase gas chromatography or crystallizing the compound in a chiral solvent. Stereoselective syntheses, a chemical or enzymatic reaction in which a single reactant forms an unequal mixture of stereoisomers during the creation of a new stereocenter or during the transformation of a pre-existing one, are well known in the art. Stereoselective syntheses encompass both enantio- and diastereoselective transformations. See, for example, Carreira and Kvaemo, Classics in Stereoselective Synthesis, Wiley-VCH: Weinheim, 2009.

Geometric isomers, resulting from the arrangement of substituents around a carbon-carbon double bond or arrangement of substituents around a cycloalkyl or heterocyclic ring, can also exist in the compounds of the present disclosure. The symbol=denotes a bond that may be a single, double or triple bond as described herein. Substituents around a carbon-carbon double bond are designated as being in the “Z” or “E” configuration, where the terms “Z” and “E” are used in accordance with IUPAC standards. Unless otherwise specified, structures depicting double bonds encompass both the “E” and “Z” isomers.

Substituents around a carbon-carbon double bond alternatively can be referred to as “cis” or “trans,” where “cis” represents substituents on the same side of the double bond and “trans” represents substituent on opposite sides of the double bond. The arrangement of substituents around a carbocyclic ring can also be designated as “cis” or “trans.” The term “cis” represents substituents on the same side of the plane of the ring and the term “trans” represents substituents on opposite sides of the plane of the ring. Mixtures of compound wherein the substituents are disposed on both the same and opposite sides of the plane of the ring are designated “cis/trans.”

Singular articles such as “a,” “an” and “the” and similar referents in the context of describing the elements are to be construed to cover both the singular and the plural, unless otherwise indicated herein or clearly contradicted by context. Recitation of ranges of values herein are merely intended to serve as a shorthand method of referring individually to each separate value falling within the range, including the upper and lower bounds of the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (i.e., “such as”) provided herein, is intended merely to better illuminate the embodiments and does not pose a limitation on the scope of the claims unless otherwise stated.

In some embodiments, where the use of the term “about” is before a quantitative value, the present disclosure also includes the specific quantitative value itself, unless specifically stated otherwise. As used herein, the term “about” refers to a 10% variation from the nominal value unless otherwise indicated or inferred. Where a percentage is provided with respect to an amount of a component or material in a composition, the percentage should be understood to be a percentage based on weight, unless otherwise stated or understood from the context.

Where a molecular weight is provided and not an absolute value, for example, of a polymer, then the molecular weight should be understood to be an average molecule weight, unless otherwise stated or understood from the context.

It should be understood that the order of steps or order for performing certain actions is immaterial so long as the present disclosure remain operable. Moreover, two or more steps or actions can be conducted simultaneously.

A dash (“—”) symbol that is not between two letters or symbols refers to a point of bonding or attachment for a substituent. For example, —NH₂ is attached through the nitrogen atom.

The terms “active agent,” “drug,” “pharmacologically active agent,” and “active pharmaceutical ingredient” are used interchangeably to refer to a compound or composition which, when administered to a subject, induces a desired pharmacologic or physiologic effect by local or systemic action or both.

The terms “individual,” “host,” and “subject,” are used interchangeably, and refer to an animal, including, but not limited to, human and non-human primates, including simians and humans; rodents, including rats and mice; bovines; equines; ovines; felines; canines; and the like. “Mammal” means a member or members of any mammalian species, and includes, by way of example, canines, felines, equines, bovines, ovines, rodentia, etc. and primates, i.e., non-human primates, and humans. Non-human animal models, i.e., mammals, non-human primates, murines, lagomorpha, etc. may be used for experimental investigations.

“Patient” refers to a human subject.

The terms “treating,” “treatment,” and the like, refer to obtaining a desired pharmacologic and/or physiologic effect, such as reduction of one or more symptoms of the disease or disorder. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease or a symptom of a disease from occurring in a subject which may be predisposed to the disease but has not yet been diagnosed as having it (i.e., including diseases that may be associated with or caused by a primary disease); (b) inhibiting the disease, i.e., arresting its development; and (c) relieving the disease, i.e., causing regression of the disease (i.e., reduction in pain or other symptom).

The term “amelioration” or any grammatical variation thereof (e.g., ameliorate, ameliorating, and amelioration etc.), includes, but is not limited to, delaying the onset, or reducing the severity of a disease or condition (e.g., diarrhea, bacteremia and/or endotoxemia). Amelioration, as used herein, does not require the complete absence of symptoms.

The term “pharmaceutically acceptable salt” refers to a salt which is acceptable for administration to a subject. It is understood that such salts, with counter ions, will have acceptable mammalian safety for a given dosage regime. Such salts can also be derived from pharmaceutically acceptable inorganic or organic bases and from pharmaceutically acceptable inorganic or organic acids, and may comprise organic and inorganic counter ions.

The neutral forms of the compounds described herein may be converted to the corresponding salt forms by contacting the compound with a base or acid and isolating the resulting salts.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like.

Other examples of salts include anions of the compounds of the present disclosure compounded with a suitable cation such as N⁺, NH₄⁺, and NW₄⁺ (where W can be a C₁-C₈ alkyl group), and the like. For therapeutic use, salts of the compounds of the present disclosure can be pharmaceutically acceptable. However, salts of acids and bases that are non-pharmaceutically acceptable may also find use, for example, in the preparation or purification of a pharmaceutically acceptable compound.

Compounds included in the present compositions that are basic in nature are capable of forming a wide variety of salts with various inorganic and organic acids. The acids that can be used to prepare pharmaceutically acceptable acid addition salts of such basic compounds are those that form non-toxic acid addition salts, i.e., salts containing pharmacologically acceptable anions, including but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts.

Compounds included in the present compositions that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

Compounds included in the present compositions that include a basic or acidic moiety can also form pharmaceutically acceptable salts with various amino acids. The compounds of the disclosure can contain both acidic and basic groups; for example, one amino and one carboxylic acid group. In such a case, the compound can exist as an acid addition salt, a zwitterion, or a base salt.

The phrase “therapeutically effective amount” refers to the amount of a compound that, when administered to a mammal or other subject for treating a disease, condition, or disorder, is sufficient to affect such treatment for the disease, condition, or disorder. The “therapeutically effective amount” will vary depending on the compound, the disease and its severity and the age, weight, etc., of the subject to be treated.

The terms “pharmaceutically acceptable excipient,” “pharmaceutically acceptable diluent,” “pharmaceutically acceptable carrier,” and “pharmaceutically acceptable adjuvant” are used interchangeably and refer to an excipient, diluent, carrier, or adjuvant that is useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. The phrase “pharmaceutically acceptable excipient” includes both one and more than one such excipient, diluent, carrier, and/or adjuvant.

The term “pharmaceutical composition” is meant to encompass a composition suitable for administration to a subject, such as a mammal, especially a human. In general a “pharmaceutical composition” is sterile, and preferably free of contaminants that are capable of eliciting an undesirable response within the subject (i.e., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intracheal, intramuscular, subcutaneous, and the like.

As used herein, the term “sustained release”, “delayed release”, and “controlled release” refer to prolonged or extended release of the therapeutic agent or API of the pharmaceutical formulation. These terms may further refer to composition which provides prolonged or extended duration of action, such as pharmacokinetics (PK) parameters of a pharmaceutical composition comprising a therapeutically effective amount of the active pharmaceutical ingredient as described herein.

Generally, reference to or depiction of a certain element such as hydrogen or H is meant to include all isotopes of that element. For example, if an R group is defined to include hydrogen or H, it also includes deuterium and tritium. Compounds comprising radioisotopes such as tritium, ¹⁴C, ³²P and ³⁵S are thus within the scope of the present technology. Procedures for inserting such labels into the compounds of the present technology will be readily apparent to those skilled in the art based on the disclosure herein.

Unless the specific stereochemistry is expressly indicated, all chiral, diastereomeric, and racemic forms of a compound are intended. Thus, compounds described herein include enriched or resolved optical isomers at any or all asymmetric atoms as are apparent from the depictions. Racemic mixtures of enantiomers, and enantio-enriched stereomeric mixtures comprising of enantiomers, as well as the individual optical isomers can be isolated or synthesized so as to be substantially free of their enantiomeric or diastereomeric partners, and these stereoisomers are all within the scope of the present technology.

The compounds described herein may exist as solvates, especially hydrates, and unless otherwise specified, all such solvates and hydrates are intended. Hydrates may form during manufacture of the compounds or compositions comprising the compounds, or hydrates may form over time due to the hygroscopic nature of the compounds. Compounds of the present technology may exist as organic solvates as well, including DMF, ether, and alcohol solvates, among others. The identification and preparation of any particular solvate is within the skill of the ordinary artisan of synthetic organic or medicinal chemistry.

As described herein, the text refers to various embodiments of the present compounds, compositions, and methods. The various embodiments described are meant to provide a variety of illustrative examples and should not be construed as descriptions of alternative species. Rather, it should be noted that the descriptions of various embodiments provided herein may be of overlapping scope. The embodiments discussed herein are merely illustrative and are not meant to limit the scope of the present technology.

### 5.5 Exemplary Embodiments

As described herein, the text refers to various embodiments of the present compounds, compositions, and methods. The various embodiments described are meant to provide a variety of illustrative examples and should not be construed as descriptions of alternative species. Rather, it should be noted that the descriptions of various embodiments provided herein may be of overlapping scope. The embodiments discussed herein are merely illustrative and are not meant to limit the scope of the present technology.

Notwithstanding the appended claims, aspects of the present disclosure are illustrated by the following clauses.

Clause 1. A substantially optically pure (+) stereoisomer of a compound of formula (I):

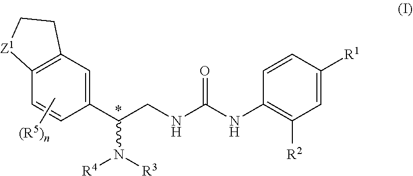

or a pharmaceutically acceptable salt thereof,
wherein:

Z¹ is selected from —O— and —NR⁶—, wherein R⁶ is selected from —H and optionally substituted (C₁-C₃)alkyl;

R¹, R² and each R⁵ are independently selected from H, halogen, OH, (C₁-C₃)alkyl, substituted (C₁-C₃)alkyl, (C₁-C₃)alkoxy, substituted (C₁-C₃)alkoxy; and

R³ and R⁴ are independently selected from —H and optionally substituted (C₁-C₃)alkyl; and

n is 0 to 3.7.

Clause 2. A substantially optically pure (−) stereoisomer of a compound of formula (I):

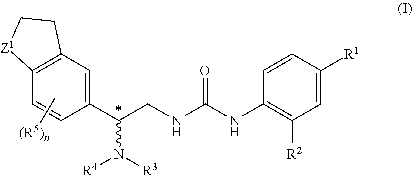

or a pharmaceutically acceptable salt thereof,
wherein:

Z¹ is selected from —O— and —NR⁶—, wherein R⁶ is selected from —H and optionally substituted (C₁-C₃)alkyl;

R¹, R² and each R⁵ are independently selected from H, halogen, OH, (C₁-C₃)alkyl, substituted (C₁-C₃)alkyl, (C₁-C₃)alkoxy, substituted (C₁-C₃)alkoxy; and

R³ and R⁴ are independently selected from —H and optionally substituted (C₁-C₃)alkyl; and

n is 0 to 3.

Clause 3. The stereoisomer of clause 1 or 2, wherein Z¹ is —NCH₃—.

Clause 4. The stereoisomer of any one of clauses 1 to 3, wherein:

R¹ is selected from —H and halogen; and

R² is selected from —H and optionally substituted (C₁-C₃)alkyl.

Clause 5 The stereoisomer of clause 4, wherein:

R¹ is halogen; and

R² is —H.

Clause 6. The stereoisomer of any one of clauses 1 to 5, wherein R³ and R⁴ are each (C₁-C₃)alkyl (e.g., methyl).

Clause 7. The stereoisomer of clause 1 or 2, wherein Z¹ is —O—.

Clause 8. The stereoisomer of clause 7, wherein;

R¹ is selected from —H and halogen; and

R² is selected from —H and optionally substituted (C₁-C₃)alkyl.

Clause 9. The stereoisomer of clause 8, wherein:

R¹ is —H; and

R² is optionally substituted (C₁-C₃)alkyl.

Clause 10. The stereoisomer of any one of clauses 7 to 9, wherein R³ and R⁴ are each (C₁-C₃)alkyl (e.g., methyl).

Clause 11. A substantially optically pure (+) stereoisomer of a compound having the structure:

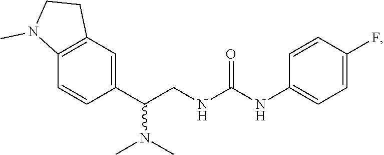 [SMILES: CN1CCc2cc(C(CNC(=O)Nc3ccc(F)cc3)N(C)C)ccc21]

or a pharmaceutically acceptable salt thereof.

Clause 12. A substantially optically pure (−) stereoisomer of a compound having the structure:

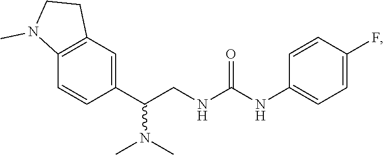 [SMILES: CN1CCc2cc(C(CNC(=O)Nc3ccc(F)cc3)N(C)C)ccc21]

or a pharmaceutically acceptable salt thereof.

Clause 13. A substantially optically pure (+) stereoisomer of a compound of the following structure:

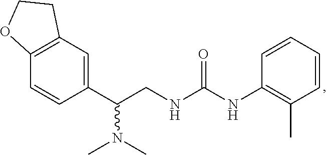 [SMILES: Cc1ccccc1NC(=O)NCC(c1ccc2c(c1)CCO2)N(C)C]

or a pharmaceutically acceptable salt thereof.

Clause 14. A substantially optically pure (−) stereoisomer of a compound of the following structure:

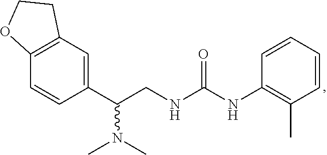 [SMILES: Cc1ccccc1NC(=O)NCC(c1ccc2c(c1)CCO2)N(C)C]

or a pharmaceutically acceptable salt thereof.

Clause 15. A substantially optically pure (+) stereoisomer of a compound of formula (II):

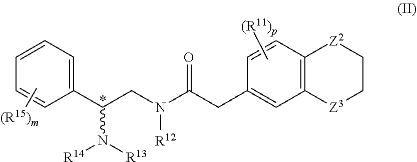

or a stereoisomer thereof, or a pharmaceutically acceptable salt thereof,
wherein:

Z² and Z³ are independently selected from —O— and —NR⁶—, wherein R¹⁶ is selected from —H and optionally substituted (C₁-C₃)alkyl;

R¹², R¹³ and R¹⁴ are independently selected from —H and optionally substituted (C₁-C₃)alkyl;

each R¹¹ and each R¹⁵ are independently selected from —H, -halogen, —OH, —CF₃, —OCF₃, —CN, —NH₂, —NO₂, optionally substituted (C₁-C₆)alkyl, optionally substituted (C₁-C₆)alkoxy, —C(O)OR¹², —C(O)NHR¹², —SO₂NHR¹², —NR¹³R¹⁴, —NHC(O)R¹², and —SO₃H;

m is 0 to 5; and

p is 0 to 3.

Clause 16. A substantially optically pure (−) stereoisomer of a compound of formula (II):

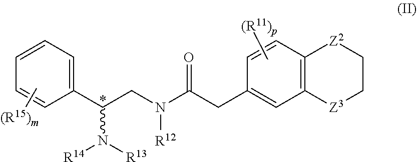

or a stereoisomer thereof, or a pharmaceutically acceptable salt thereof,
wherein:

Z² and Z³ are independently selected from —O— and —NR¹⁶—, wherein R¹⁶ is selected from —H and optionally substituted (C₁-C₃)alkyl;

R¹², R¹³ and R¹⁴ are independently selected from —H and optionally substituted (C₁-C₃)alkyl;

each R¹¹ and each R¹⁵ are independently selected from —H, -halogen, —OH, —CF₃, —OCF₃, —CN, —NH₂, —NO₂, optionally substituted (C₁-C₆)alkyl, optionally substituted (C₁-C₆)alkoxy, —C(O)OR¹², —C(O)NHR¹², —SO₂NHR¹², —NR¹³R¹⁴, —NHC(O)R¹², and —SO₃H;

m is 0 to 5; and

p is 0 to 3.

Clause 17. The stereoisomer of clause 15 or 16, wherein R¹² is —H.

Clause 18. The stereoisomer of any one of clauses 15 to 17, wherein the compound is of formula (III):

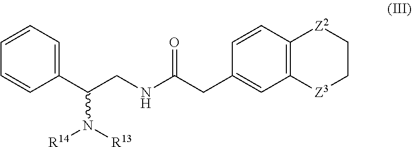

or a pharmaceutically acceptable salt thereof.

Clause 19. The stereoisomer of clause 18, wherein Z² is —O—.

Clause 20. The stereoisomer of clause 18 or 19, wherein Z³ is —O—.

Clause 21. The stereoisomer of any one of clauses 18 to 20, wherein R¹³ and R¹⁴ are each (C₁-C₃)alkyl (e.g., methyl).

Clause 22. A substantially optically pure (+) stereoisomer of a compound of the following structure:

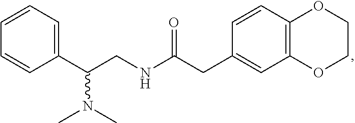 [SMILES: CN(C)C(CNC(=O)Cc1ccc2c(c1)OCCO2)c1ccccc1]

or a pharmaceutically acceptable salt thereof.

Clause 23. A substantially optically pure (−) stereoisomer of a compound of the following structure:

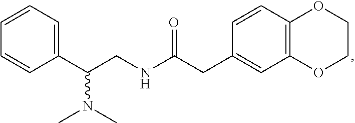 [SMILES: CN(C)C(CNC(=O)Cc1ccc2c(c1)OCCO2)c1ccccc1]

or a pharmaceutically acceptable salt thereof.

Clause 24. The stereoisomer of any one of clauses 1 to 23, having an enantiomeric excess of 80% or more (e.g., 85% or more).

Clause 25. The stereoisomer of clause 24, having an enantiomeric excess of 90% or more (e.g., 95% or more).

Clause 26. The stereoisomer of clause 25, having an enantiomeric excess of 98% or more (e.g., 99% or more).

Clause 27. A pharmaceutical composition, comprising:

(i) a stereoisomer of any one of clauses 1 to 26; and

(ii) a pharmaceutically acceptable excipient.

Clause 28. A pharmaceutical composition, comprising:

(i) a compound of formula (I):

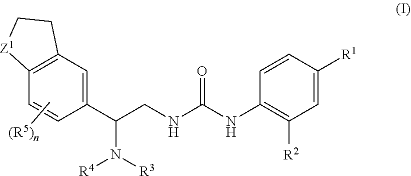

or a stereoisomer thereof, or a pharmaceutically acceptable salt thereof,
wherein:

Z¹ is selected from —O— and —NR⁶—, wherein R⁶ is selected from —H and optionally substituted (C₁-C₃)alkyl;

R¹, R² and each R⁵ are independently selected from H, halogen, OH, (C₁-C₃)alkyl, substituted (C₁-C₃)alkyl, (C₁-C₃)alkoxy, substituted (C₁-C₃)alkoxy; and

R³ and R⁴ are independently selected from —H and optionally substituted (C₁-C₃)alkyl; and

n is 0 to 3; and

(ii) a pharmaceutically acceptable excipient.

Clause 29. The pharmaceutical composition of clause 28, wherein Z¹ is —NCH₃—.

Clause 30. The pharmaceutical composition of clause 28 or 29, wherein:

R¹ is selected from —H and halogen; and

R² is selected from —H and optionally substituted (C₁-C₃)alkyl.

Clause 31. The pharmaceutical composition of clause 30, wherein:

R¹ is halogen; and

R² is —H.

Clause 32. The pharmaceutical composition of any one of clauses 28 to 31, wherein R³ and R⁴ are each (C₁-C₃)alkyl (e.g., methyl)

Clause 33. The pharmaceutical composition of clause 32, wherein the compound is

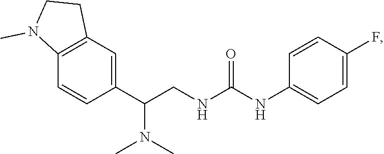 [SMILES: CN1CCc2cc(C(CNC(=O)Nc3ccc(F)cc3)N(C)C)ccc21]

or a stereoisomer thereof, or a pharmaceutically acceptable salt thereof.

Clause 34. The pharmaceutical composition of clause 28, wherein Z¹ is —O—.

Clause 35. The pharmaceutical composition of clause 34, wherein;

R¹ is selected from —H and halogen; and

R² is selected from —H and optionally substituted (C₁-C₃)alkyl.

Clause 36. The pharmaceutical composition of clause 35, wherein:

R¹ is —H; and

R² is optionally substituted (C₁-C₃)alkyl.

Clause 37. The pharmaceutical composition of any one of clauses 34 to 36, wherein R³ and R⁴ are each (C₁-C₃)alkyl (e.g., methyl).

Clause 38. The pharmaceutical composition of clause 37, wherein the compound is

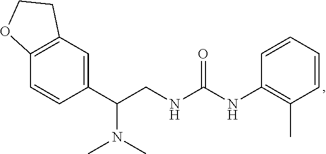 [SMILES: Cc1ccccc1NC(=O)NCC(c1ccc2c(c1)CCO2)N(C)C]

or a stereoisomer thereof, or a pharmaceutically acceptable salt thereof.

Clause 39. A pharmaceutical composition, comprising:

(i) a compound of formula (II):

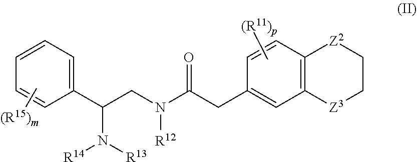

or a stereoisomer thereof, or a pharmaceutically acceptable salt thereof,
wherein:

Z² and Z³ are independently selected from —O— and —NR¹⁶—, wherein R¹⁶ is selected from —H and optionally substituted (C₁-C₃)alkyl;

R¹², R¹³ and R¹⁴ are independently selected from —H and optionally substituted (C₁-C₃)alkyl;

each R¹¹ and each R¹⁵ are independently selected from —H, -halogen, —OH, —CF₃, —OCF₃, —CN, —NH₂, —NO₂, optionally substituted (C₁-C₆)alkyl, optionally substituted (C₁-C₆)alkoxy, —C(O)OR¹², —C(O)NHR¹², —SO₂NHR¹², —NR¹³R¹⁴, —NHC(O)R¹², and —SO₃H;

m is 0 to 5; and

p is 0 to 3; and
(ii) a pharmaceutically acceptable excipient.

Clause 40. The pharmaceutical composition of clause 39, wherein R¹² is —H.

Clause 41. The pharmaceutical composition of clause 39 or 40, wherein the compound is of formula (III):

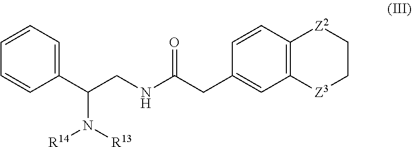

or a stereoisomer thereof, or a pharmaceutically acceptable salt thereof.

Clause 42. The pharmaceutical composition of clause 41, wherein Z² is —O—.

Clause 43. The pharmaceutical composition of clause 41 or 42, wherein Z³ is —O—.

Clause 44. The pharmaceutical composition of any one of clauses 41 to 43, wherein R¹³ and R¹⁴ are each (C₁-C₃)alkyl (e.g., methyl).

Clause 45. The pharmaceutical composition of any one of clauses 41 to 44, wherein the compound is

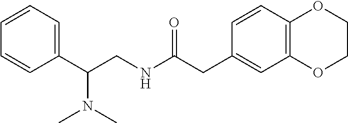 [SMILES: CN(C)C(CNC(=O)Cc1ccc2c(c1)OCCO2)c1ccccc1]

or a stereoisomer thereof, or a pharmaceutically acceptable salt thereof.

Clause 46. A method of modulating the activity of an opioid receptor, comprising contacting a biological system with a stereoisomer according to any one of clauses 1 to 27, or a pharmaceutical composition according to any one of clauses 28 to 45.

Clause 47. The method of clause 46, wherein the biological system is in vitro.

Clause 48. The method of clause 46, wherein the biological system is in vivo.

Clause 49. A method of treating pain, comprising administering to a subject having pain an effective amount of a pharmaceutical composition according to any one of clauses 28 to 45, to treat the subject for pain.

Clause 50. The method of any one of clauses 46 to 49, wherein the compound is

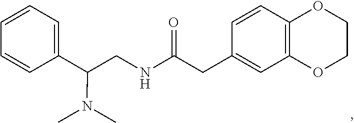 [SMILES: CN(C)C(CNC(=O)Cc1ccc2c(c1)OCCO2)c1ccccc1]

or a stereoisomer thereof, or a pharmaceutically acceptable salt thereof.

Clause 51. The method of any one of clauses 46 to 49, wherein the compound is

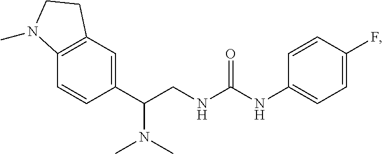 [SMILES: CN1CCc2cc(C(CNC(=O)Nc3ccc(F)cc3)N(C)C)ccc21]

or a stereoisomer thereof, or a pharmaceutically acceptable salt thereof.

Clause 52. The method of any one of clauses 46 to 49, wherein the compound is

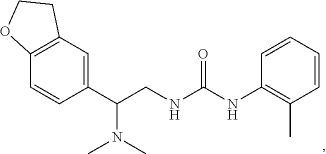 [SMILES: Cc1ccccc1NC(=O)NCC(c1ccc2c(c1)CCO2)N(C)C]

or a stereoisomer thereof, or a pharmaceutically acceptable salt thereof.

Clause 53. A stereoisomer compound for use in treating pain, wherein the stereoisomer is according to any one of claims 1 to 27.

Clause 54. A pharmaceutical composition for use in treating pain, wherein the pharmaceutical composition is according to any one of clauses 28 to 45.

Clause 55. Use of a stereoisomer according to any one of clauses 1 to 27, or a pharmaceutical composition according to any one of clauses 28 to 45 in the manufacture of a medicament for treating pain.

### 6. EXAMPLES

The following examples are offered to illustrate the present disclosure and are not to be construed in any way as limiting the scope of the present technology. Any methods that are functionally equivalent are within the scope of the present technology. Various modifications of the present technology in addition to those described herein will become apparent to those skilled in the art from the foregoing description and accompanying figures. Such modifications fall within the scope of the appended claims.

Unless otherwise stated, all temperatures are in degrees Celsius. Efforts have been made to ensure accuracy with respect to numbers used (e.g., amounts, temperatures, etc.), but some experimental errors and deviation should be allowed for.

### Example 1—In Vitro Assays for Determining Selective μ-Opioid Receptor Agonists

### Introduction

Drugs that target opioid receptors are essential to managing severe pain. However, classical opioid drugs are associated with side effects including gastrointestinal (GI) tract dysfunction and respiratory system depression. Functional selectivity opioid receptor agonists, which selectively activate the G protein pathway rather than the β-arrestin pathway, can overcome the side effects of drugs. TRV130 (oliceridine) and PZM21 are known as μ-opioid receptor agonists that failed during translational research. One of the major reasons for the failure is that overestimated data was used to compare the efficacy between different signalings. We discovered compounds that overcome the overestimated data issue and can exhibit a better pharmacological effect on the life-organ system.

### Materials and Methods

TRV130 (Oliceridine) is purchased from AdooQ (CA, US). PZM21 was purchased from MedKOO bioscience (NC, US). SNC80 and [D-Ala², NMe-Phe⁴, Gly-ol⁵]-enkephalin (DAMGO) were purchased from Tocris (Bristol, UK). Candidate compounds were purchased from MolPort (Riga, Latvia). β-Funaltrexamine hydrochloride (β-FNA) and β-Chlornaltrexamine dihydrochloride (β-CNA) were purchased from Merck (Darmstadt, Germany). PathHunter β-arrestin assay kit was purchased from DiscoverX (CA, USA). cAMP assay kit was purchased from Cisbio (codolet, France).

### Cell Line Generation

Human embryonic kidney 293 (HEK-293) cells which transfected overexpress (3-arrestin2 fused β-galactosidase fragment were purchased from DiscoverX. Human OPRM1 gene (NM 000914.3, encoding human μ-opioid receptor, OPRM) and Human OPRD1 gene (NM 000911.3, encoding human δ-opioid receptor, OPRD) were fused to a pCMV-ProLink plasmid which contains complementary β-galactosidase fragment purchased from DiscoverX. To generate human OPRM1 or OPRD1 expression cell lines, HEK-293 cells were transfected to prepared plasmid and grown in DMEM medium with 10% fetal bovine serum, 150 μg/mL Hygromycin B and 500 μg/mL G418.

### β-Arrestin 2 Assay

The PathHunter enzyme assay was performed to measure the agonist's capability of β-arrestin 2 pathway activation. When agonists bind to G protein-coupled receptor (GPCR), the receptor pathway is sequentially activated from G protein to β-arrestin 2 recruitment.

Because endogenous β-arrestin 2 and C-terminal of GPCR have a fragment of β-galactosidase, recruitment of β-arrestin 2 to GPCR forms the whole β-galactosidase. The functionality of the whole β-galactosidase is detected by chemiluminescence (FlexStation3, Molecular Devices).

Cyclic Adenosine Monophosphate (cAMP) Accumulation

The agonist's efficacy to the G protein pathway was determined by changes in the cAMP level using the Cisbio cAMP detection kit. The cells used in cAMP assays are the same cell line as used in the β-arrestin 2 assays. The human opioid receptor activates the G_{αi} so intracellular cAMP accumulation is inhibited. Cisbio detection kit uses labeled cAMP and antibody. This system proceeds to compete with endogenous cAMP. If G_{αi} is activated, intracellular cAMP decreases. This assures that labeled-cAMP binds more with the antibody so that time-resolved fluorescence increases.

### Dominant Assay

To confirm the agonist's efficacy in a nature-like system, we need inactivation of target receptors on the cell membrane. Most cell lines have an abundant target receptor population (also known as spare receptors). Therefore, agonists have more opportunity to constitute agonist-receptor conformation. In this environment, not only full agonists but also partial agonists can reach maximal response.

If the partial agonist is selected as a full agonist, translational in vivo research will likely fail. Therefore, when judging whether a compound is a full agonist or partial agonist, it is important to confirm whether the result is due to the spare receptors or not.

Non-competitive, irreversible antagonists can be applicable to judge the pharmacological character of agonists because they permanently decrease the receptor population in the cell line system. β-FNA or β-CNA is used as a non-competitive and irreversible antagonist for OPRM, OPRD. Before cAMP assay, cells were incubated with β-FNA or β-CNA for 40 minutes. Afterwards, the procedure is the same.

If a partial agonist compound acts as a full agonist due to receptor population (pseudo full agonist), it cannot sustain maximal response with reduced receptor population. However, a full agonist compound maintains their maximal response even though increased EC₅₀.

### Example 2—Identification of μ-Opioid Receptor Agonists

### TRV130, PZM21

A compound which precisely modulates cell signaling is expected to draw a better response than classical agonist or antagonist. A GPCR related target which has sequential and diversified signaling pathway is more suitable to precise modulation, e.g., via a “biased agonist”.

TRV130 (oliceridine) is an μ-opioid bias agonist of the G protein pathway. TRV130 has an equivalent effect on cAMP accumulation (G protein pathway). However, TRV130 shows reduced efficacy on the β-arrestin2 pathway. See e.g., DeWire, et al. (“A G protein-biased ligand at the mu-opioid receptor is potently analgesic with reduced gastrointestinal and respiratory dysfunction compared with morphine.” J Pharmacol Exp Ther 2013, 344 (3), 708-17). However, TRV130 did not perform as a biased agonist in clinical trials, and showed a similar level of benefit and side effects to morphine.

PZM21 was the second drug in the pipeline of biased agonists and is used as a reference agonist in many studies. PZM21 is reported to be more efficacious on the G protein pathway than TRV130. Although, PZM21 was reported to show reduced opioid receptor-dependent addiction, it was shown PZM21 is not a biased agonist and is a low efficacy agonist that can induce respiratory depression. See e.g., Hill et al. (“The novel mu-opioid receptor agonist PZM21 depresses respiration and induces tolerance to antinociception.” Br J Pharmacol 2018, 175 (13), 2653-2661).

These drugs were assessed by a flawed pharmacological analysis due to amplificated in vitro results.

Assays of G protein activity are commonly confounded by the presence of spare receptors. In the presence of high receptor reserve, most agonists reach a similar maximal response. The problem is most cell lines that are used for in vitro assays contain a lot of spare receptors.

In the GPCR signaling pathway, proximity from the receptor is related to the amplification level. The signal downstream is overactivated than the receptor-agonist binding effect due to nonspecific intracellular molecules. Spare receptors and downstream overestimation make tend to prevent a straightforward determination of relative efficacy.

Regardless of the model, the identification of biased agonists requires system-independent data analysis. The standard for bias quantification is the operational model of agonism which mathematically accounts for the distinct pharmacological parameters of affinity and efficacy. However, sometimes the model produces system-dependent data.

Although the operational model has the benefit to calculate the receptor-agonist effect, binding kinetic including downstream signaling processes is not accounted for in the model. While the cAMP is the second molecule in the G protein pathway, β-arrestin directly binds to the receptor. Therefore, results from cAMP assay are always overestimated than 3-arrestin assay. If simply compare data, it makes decisions that most compounds including TRV130 and PZM21 are classified as G protein-biased agonists even though the results are due to amplification.

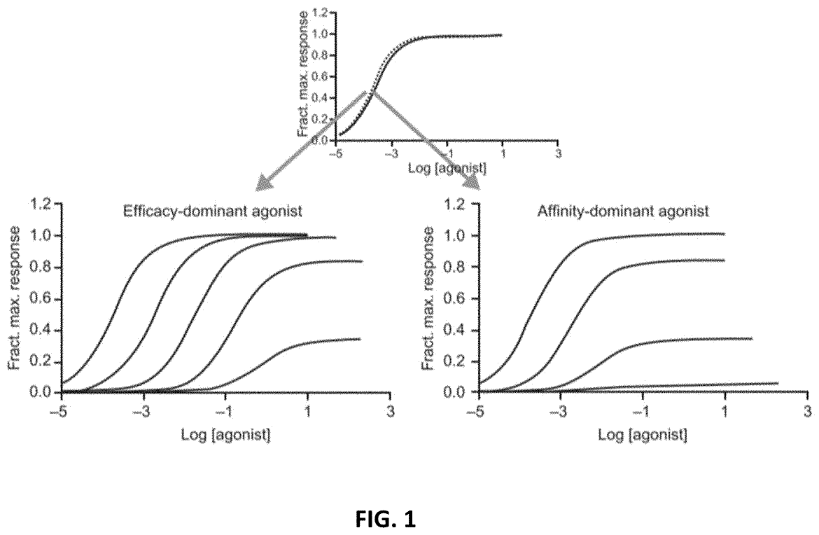

See e.g., FIG. 1 of Gillis et al. (“Low intrinsic efficacy for G protein activation can explain the improved side effect profiles of new opioid agonists.” Sci Signal 2020, 13 (625)). For example, in pathways with very limited signal amplification due to direct proximity to receptor activation (Nb33 recruitment and mGsi recruitment) or with very limited receptor reserve from partially irreversible antagonism (GIRK activation), partial agonists had lower efficacy and potency than in highly amplified pathways (Gui2 activation and AC inhibition). However, despite these differences, estimates of relative efficacy for activating G proteins were remarkably consistent across assays of receptor and G protein activation.

The problems described above can be handled by using cells treated by irreversible antagonists. Using this methodology, we selected and confirmed activity of exemplary compounds that have better pharmacological profiles than TRV130 and PZM21.

### Results

### Library Screening

By using Computer-Aided Drug Design (CADD), an optimal library consisting of virtual hit candidate compounds was created. These compounds are readily synthesized according to standard synthetic methods known in the art. In some cases, the compounds are commercially available.

cAMP and β-arrestin assays described above were conducted to decide whether candidate compounds were μ- and/or δ-opioid receptor bias agonists for the G-protein pathway. All assays produced the concentration-response curve, and the curve data was validated by a quality control factor named Z′ factor. Because half-maximal effect concentration (EC₅₀) depends on the population of receptors, the maximal asymptote of the concentration curve was used. Efficacy data of candidates was normalized by comparison to reference agonists DAMGO and SNC80 for OPRM and OPRD.

The hill slope describes the steepness of the curve. A standard sigmoidal concentration-response curve has a hill slope of 1.0 which means that agonist and signaling molecules have a canonical reaction. When a compound has a hill slope less or greater than 1.0, said compound was considered to trigger a cooperative binding state. We set the appropriate interval of hill slope as 0.8<n<1.5 which maintains the sigmoidal curve shape.

Three compounds of the library that were obtained from commercial sources exhibited desirable activity in the assays, include:
compound 1: 2-(2,3-dihydro-1,4-benzodioxin-6-yl)-N-[2-(dimethylamino)-2-phenylethyl]acetamide (CAS number 1110883-26-5);
compound 2: 3-[2-(dimethylamino)-2-(1-methyl-2,3-dihydro-1H-indol-5-yl)ethyl]-1-(4-fluorophenyl)urea (CAS number 1172882-21-1); and
compound 3: 3-[2-(2,3-dihydro-1-benzofuran-5-yl)-2-(dimethylamino)ethyl]-1-(2-methylphenyl)urea (CAS number 1428367-46-7).

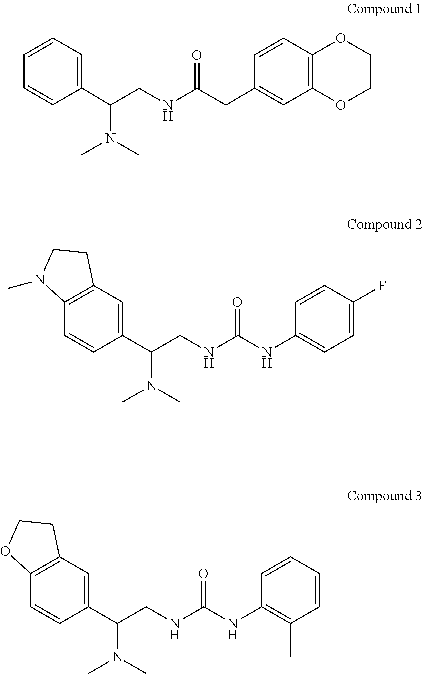 [SMILES: CN(C)C(CNC(=O)Cc1ccc2c(c1)OCCO2)c1ccccc1 ; CN1CCc2cc(C(CNC(=O)Nc3ccc(F)cc3)N(C)C)ccc21 ; Cc1ccccc1NC(=O)NCC(c1ccc2c(c1)CCO2)N(C)C]

Table 1 shows in vitro results of cAMP and β-arrestin assays of exemplary compound 1, compound 2, and compound 3, and reference compounds TRV130 (oliceridine) and PZM21.

TABLE 1
cAMP and β-arrestin assay results

| | Compound | | | | |
|---|---|---|---|---|---|
| ID | 1 | 2 | 3 | TRV130 | PZM21 |
| OPRM Gi Emax, % | 102.3 | 109.13 | 112.22 | 100.83 | 91.3 |
| OPRM Gi EC₅₀, uM | 0.73 | 1.88 | 1.29 | 0.03 | 0.06 |
| OPRM arrestin Emax, % | 12.86 | 15.26 | 12.21 | 2.41 | 2.68 |
| OPRM Gi Hill slope | 0.99 | 0.94 | 1.48 | 0.35 | 0.72 |
| OPRD Gi Emax, % | 88.93 | 92.39 | 94.63 | 63.41 | 98.3 |
| OPRD Gi EC₅₀, uM | 4.16 | 1.05 | 8.37 | 2.04 | 0.23 |
| OPRD arrestin Emax, % | 7.37 | 4.66 | 1.99 | 9.60 | 3.41 |
| OPRD Gi Hill slope | 1.39 | 1.44 | 1.16 | 1.28 | 0.83 |

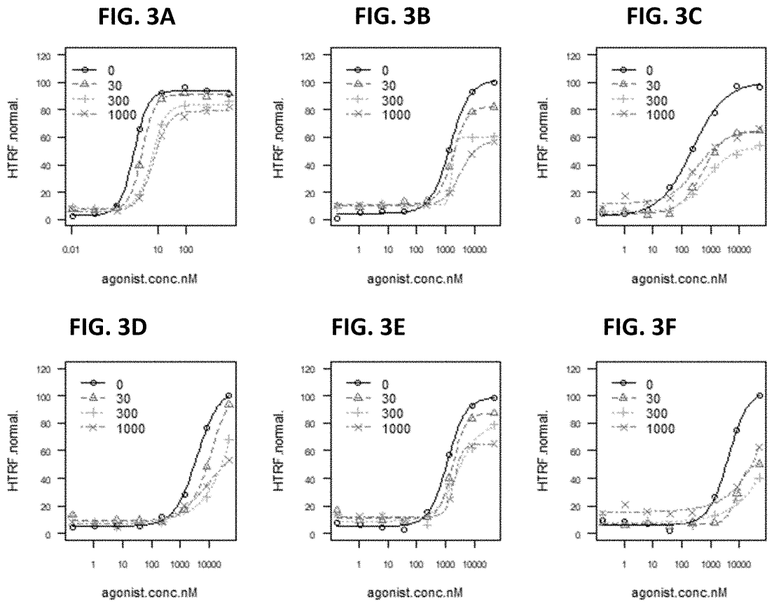

As shown in Table 1, compounds 1-3 caused full Gi pathway agonism on OPRM and OPRD, but with slight activation of the β-arrestin pathway, similar to TRV130 and PZM21. Although TRV130 showed full agonism on OPRM, the slope was only 0.35, suggesting that TRV130 has cooperative binding mode rather than system independent binding. The data of PZM21 suggested that it is an OPRM, OPRD dual biased agonist. Also, the concentration-response curve of OPRD was measured and is illustrated in FIG. 3A-3F.

As shown, although the concentration curve was stopped due to the X-axis limitation, the shape of the graph suggested that high concentrations of drug efficacy can reach high efficacy.

### Dominant Assay Results

Full agonists that can draw a maximal response from the cell system can be divided into two types of agonists. First, the agonists called efficacy-dominant have their effect due to the powerful intrinsic reaction between agonist-receptors. In this case, the agonists need relatively few receptors to draw effect. Second, affinity-dominant agonists have high affinity and weak intrinsic efficacy. The affinity-dominant agonists have restrictions to generate a maximal response like that cell membranes express the spare receptors or second signaling molecules induce amplificated signaling.

Overestimation due to spare receptor and signaling amplification is suggested as the reason why TRV130 and PZM21 have failed to translational research. The best way to confirm whether compound efficacies result from high intracellular efficacy or high affinity with spare receptor environment is by observing in a real-time system sequentially.

When spare receptors are inactivated by the irreversible and noncompetitive antagonists, full agonists show different pharmacological profiles. Specifically, the maximal response to the efficacy dominant agonist is more resistant to decrease spare receptors than affinity dominant agonists. Therefore, the concentration-response curve of the efficacy-dominant agonists shift to the right with sustaining maximal response. However, affinity-dominant agonists return a depressing maximal response.

FIG. 1 shows exemplary graphs of dominant assay results that exhibit the effects of decreasing receptor number on two agonists. The efficacy-dominant agonist has high efficacy (t=5000) and low affinity (K_{A}=1), while the affinity-dominant agonist has low efficacy (τ=50) and high affinity (K_{A}=0.01). Top curves show that both agonists are equi-active in a high receptor density system. However, as receptor density decreases in 10-fold increments, the curves for the efficacy-dominant agonist shift to the right but retain maximal response until a 100-fold shift is attained, while the curves to the affinity-dominant agonist show depressed maxima with any decrease in receptor number.

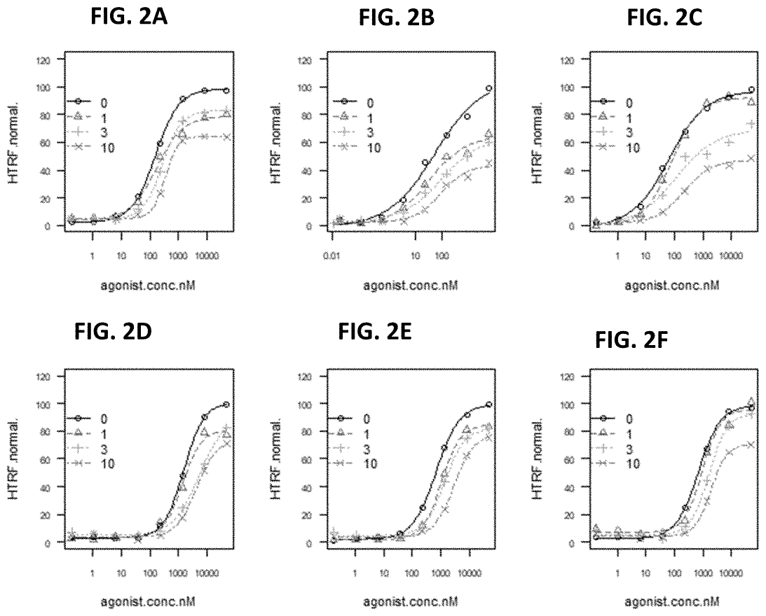

To confirm the dominant tendency of candidate compounds, β-FNA and CNA were used to decrease spare receptors of the OPRM and OPRD cell line respectively (FIGS. 2A-2F and 3A-3F). The results (FIGS. 2A-2F, where each curve represents 0-10 nM of β-FNA as indicated) showed that decreasing OPRM receptors on the cell membrane attenuates the efficacy of TRV130 (FIG. 2B) and PZM21 (FIG. 2C) on the G protein pathway. However, compounds 1-3 (FIGS. 2D-2F, respectively) were resistant to decrease spare receptors-induced tendency. This suggested that the maximal responses of candidates compounds were generated from powerful intrinsic efficacy.

During discovery, pharmacological dominance of TRV130 and PZM21 was not confirmed. Therefore, they directly compared the G protein assay results and 0-arrestin results even though the maximal response of cAMP assay (the activity of the G protein pathway) due to overestimation. Pseudo-bias to the G protein pathway due to overestimation was an important reason that TRV130 and PZM21 failed to translational research.

FIGS. 2A-2F shows the results from a dominant assay using OPRM cell lines, where each curve represents 0-10 nM of R-FNA as indicated. DAMGO is a full agonist of OPRM (FIG. 2A). The maximal responses of TRV130 (FIG. 2B) and PZM21 (FIG. 2C) are more susceptible to decreasing receptor populations than DAMGO. However, compound 1 (FIG. 2D), compound 2 (FIG. 2E), and compound 3 (FIG. 2F) showed a similar pattern as compared to DAMGO.

FIGS. 3A-3F shows the results from a dominant assay using OPRD cell line, where each curve represents 0-1000 nM of R-CNA as indicated. SNC80 is a full agonist for OPRD (FIG. 3A). The maximal responses of TRV130 (FIG. 3B), PZM21 (FIG. 3C), and exemplary compounds 1-3 (FIGS. 3D-3F, respectively) were more susceptible to decreasing receptor populations than SNC80.

In the OPRD system, TRV130 (FIG. 3B), PZM21 (FIG. 3C), and our candidates compounds 1-3 (FIGS. 3D-3F, respectively) showed less resistance to decreasing spare receptors than SNC80, a full agonist of OPRD (FIG. 3A). Because the efficacies of opioids are more related to OPRM than OPRD, the pharmacological properties in OPRM are believed to be more important than OPRD. Therefore, the candidate compounds 1-3 have a comparative advantage in terms of painkiller activity over TRV130, although they have similar pharmacological properties in OPRD. Compound 2 was demonstrated to be the preferred candidate compound due to its potency. SNC80, TRV130, PZM21, and Compound 2 produced a full concentration-response curve. However, Compound 1 and Compound 3 did not since their curve was cut due to the X-axis limitation which suggested that higher concentrations of these compounds are needed to activate receptors.

### Conclusion

Both cAMP and β-arrestin in vitro assays were conducted with various compounds to identify a superior functional selective agonist compounds for the G-protein pathway rather than the β-arrestin pathway on OPRM and OPRD. Compounds 1-3 were demonstrated to have overcome the issue of results overestimation in the in vitro OPRM system associated with known drugs of TRV130 and PZM21.

This disclosure describes efforts to discover compounds for managing pain, e.g., heat-related pain, mechanical stress-related pain, and disease-related pain. First, we constructed a computer-aided drug design chemical library containing 54 compounds. To find a superior functional selectivity agonist for the G protein pathway over the β-arrestin pathway on OPRM and OPRD, we conducted cAMP assays (one of the signaling molecules on the G protein pathway) and β-arrestin assays on selected candidate compounds.

Reference compounds TRV130 and PZM21 have an issue in that their assessments did not consider overestimated results of the cAMP assay. However, our compounds 1-3 overcome the issue by assessment using the in vitro OPRM system.

The in vivo activity of exemplary compounds of this disclosure is assessed using animal models, such as a heat-related pain model, a mechanical stress-related pain model, GI tract disorder-related model, and a respiratory depression-related model, in order to demonstrate that selected compounds show analgesic efficacy but do not induce significant respiratory system depression and GI tract disorder.

### Example 3—Investigation of Stereoisomers of Compounds 1-3

Compounds 1-3 were each separated by HPLC into two enantiomeric compounds as show below in Tables 2 and 3 below:

TABLE 2
enantiomers of Compounds 1-3

| 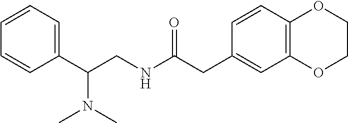 | 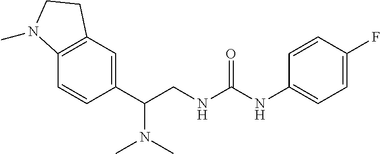 [SMILES: CN1CCc2cc(C(CNC(=O)Nc3ccc(F)cc3)N(C)C)ccc21] |
|---|---|
| Compound 1 | Compound 2 |
| Compound 4 | Compound 6 |
| (+) stereoisomer | (+) stereoisomer |
| Compound 5 | Compound 7 |
| (−) stereoisomer | (−) stereoisomer |
| | 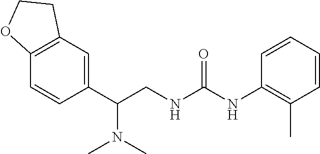 [SMILES: Cc1ccccc1NC(=O)NCC(c1ccc2c(c1)CCO2)N(C)C] |
| | Compound 3 |
| | Compound 8 |
| | (+) stereoisomer |
| | Compound 9 |
| | (−) stereoisomer |

HPLC chiral column separation conditions were as follows:

for Compound 1, CHIRALPAK AY column, 5 cm ID.×25 cm L, 10 m, mobile phase: 100% EtOH;

for Compound 2, CHIRALCEL OZ column, 2.5 cm ID.×25 cm L, 10 m, mobile phase: 60% Hexane/40% IPA; and

for Compound 3, CHIRALCEL OZ column, 2.5 cm ID.×25 cm L, 10 m, mobile phase: 75% Hexane/25% IPA.

TABLE 3
HPLC data for separation of compounds 1-3

| | HPLC Data | | |
|---|---|---|---|
| | Retention time | | % enantiomeric |
| Compound Number | (r.t.) | % area | excess (% ee) |
| Compound 1 - peak 1 | 5.941 | 99.974 | 99.95 |
| (compound 4) | | | |
| Compound 1 - peak 2 | 8.883 | 99.966 | 99.93 |
| (compound 5) | | | |
| Compound 2 - peak 1 | 4.769 | 99.580 | 99.16 |
| (compound 6) | | | |
| Compound 2 - peak 2 | 5.655 | 99.688 | 99.38 |
| (compound 7) | | | |
| Compound 3 - peak 1 | 11.916 | 99.857 | 99.17 |
| (compound 8) | | | |
| Compound 3 - peak 2 | 13.096 | 99.633 | 99.27 |
| (compound 9) | | | |

The specific optical rotations of Compounds 4-9 were measured in MeOH as shown in Table 4.

TABLE 4
specific optical rotation

| Compound | [α]₅₈₉ |
|---|---|
| 4 | +45.55 |
| 5 | −44.04 |
| 6 | +61.33 |
| 7 | −83.15 |
| 8 | +69.57 |
| 9 | −56.54 |

Each of the enantiomeric compounds were subjected to the in vitro assays as set out in Example 1 (e.g., as described herein above) and compared to their racemic parent compounds (Compounds 1-3) and TRV130 and PZM21.

The results of these assays are summarized in Tables 5-7 below, where “β-FNA (−)” refers to no addition of β-FNA and “β-FNA (+) refers to addition of β-FNA; and similarly “3 CNA (−)” refers to no addition of β-CNA and “β-CNA (+) refers to addition of β-CNA.

TABLE 5
Summary of findings for compound 1 and its enantiomers

| | | | Compound Number | | | | |
|---|---|---|---|---|---|---|---|
| | | | TRV130 | PZM21 | 1 | 4 | 5 |
| OPRM | Gi | Eₘₐₓ (%) | 89.52 | 98.27 | 101.27 | 82.79 | 102 |
| | β-FNA (−) | EC₅₀ (nM) | 120.78 | 76.56 | 2089.3 | 19970 | 292.4 |
| | Gi | Eₘₐₓ (%) | 56.33 | 68.22 | 88.23 | 24.61 | 21.91 |
| | β-FNA (+) | EC₅₀ (nM) | 214.29 | 163.31 | 5861.38 | 10914.40 | 2454.71 |
| | β-ARR2 | Eₘₐₓ (%) | 2.4 | 2.68 | 12.86 | 4.8 | 23.1 |
| OPRD | Gi | Eₘₐₓ (%) | 73.93 | 95.97 | 113.1 | 20.59 | 111 |
| | β-CNA (−) | EC₅₀ (nM) | 1.49 | 118.85 | 4.906 | N/C | 6665 |
| | Gi | Eₘₐₓ (%) | 67.24 | 58.77 | 108.4 | 17.23 | 79.38 |
| | β-CNA (+) | EC₅₀ (nM) | 1202.26 | 288.40 | 9.0 | N/C | 6095.37 |
| | β-ARR2 | Eₘₐₓ (%) | 9.6 | 3.41 | 7.37 | 2.8 | 5.7 |

N/C: not calculated by GraphPad program

TABLE 6
Summary of findings for compound 2 and its enantiomers

| | | | Compound Number | | | | |
|---|---|---|---|---|---|---|---|
| | | | TRV130 | PZM21 | 2 | 6 | 7 |
| OPRM | Gi | Eₘₐₓ (%) | 89.52 | 98.27 | 98.61 | 106.3 | 95.81 |
| | β-FNA (−) | EC₅₀ (nM) | 120.78 | 76.56 | 682.34 | 518.7 | 41.63 |
| | Gi | Eₘₐₓ (%) | 56.33 | 68.22 | 80.1 | 77.04 | 85.93 |
| | β-FNA (+) | EC₅₀ (nM) | 214.29 | 163.31 | 1174.90 | 2483.13 | 727.78 |
| | β-ARR2 | Eₘₐₓ (%) | 2.4 | 2.68 | 15.26 | 8.5 | 22.0 |
| OPRD | Gi | Eₘₐₓ (%) | 73.93 | 95.97 | 98.86 | 128.1 | 98.3 |
| | β-CNA (−) | EC₅₀ (nM) | 1.49 | 118.85 | 4.98 | 8907 | 517 |
| | Gi | Eₘₐₓ (%) | 67.24 | 58.77 | 78.46 | 119.4 | 100.2 |
| | β-CNA (+) | EC₅₀ (nM) | 1202.26 | 288.40 | 2376.84 | 48344.69 | 4897.79 |
| | β-ARR2 | Eₘₐₓ (%) | 9.6 | 3.41 | 4.66 | 10.5 | 6.4 |

TABLE 7
Summary of findings for compound 3 and its enantiomers

| | | | Compound Number | | | | |
|---|---|---|---|---|---|---|---|
| | | | TRV130 | PZM21 | 3 | 8 | 9 |
| | Gi | Eₘₐₓ (%) | 89.52 | 98.27 | 97.88 | 84.73 | 98.76 |
| OPRM | β-FNA (−) | EC₅₀ (nM) | 120.78 | 76.56 | 656.15 | 734.1 | 73.31 |
| | Gi | Eₘₐₓ (%) | 56.33 | 68.22 | 91.27 | 43.16 | 52.64 |
| | β-FNA (+) | EC₅₀ (nM) | 214.29 | 163.31 | 1581.25 | 14190.58 | 1663.41 |
| | β-ARR2 | Eₘₐₓ (%) | 2.4 | 2.68 | 12.21 | 5.4 | 22.9 |
| OPRD | Gi | Eₘₐₓ (%) | 73.93 | 95.97 | 105.5 | 95.54 | 66.82 |
| | β-CNA (−) | EC₅₀ (nM) | 1.49 | 118.85 | 4345.1 | 14970 | 2793 |
| | Gi | Eₘₐₓ (%) | 67.24 | 58.77 | 50.86 | 42.67 | 51.61 |
| | β-CNA (+) | EC₅₀ (nM) | 1202.26 | 288.40 | 10568.18 | 35727.28 | 5861.38 |
| | β-ARR2 | Eₘₐₓ (%) | 9.6 | 3.41 | 1.99 | 5.1 | 16.1 |

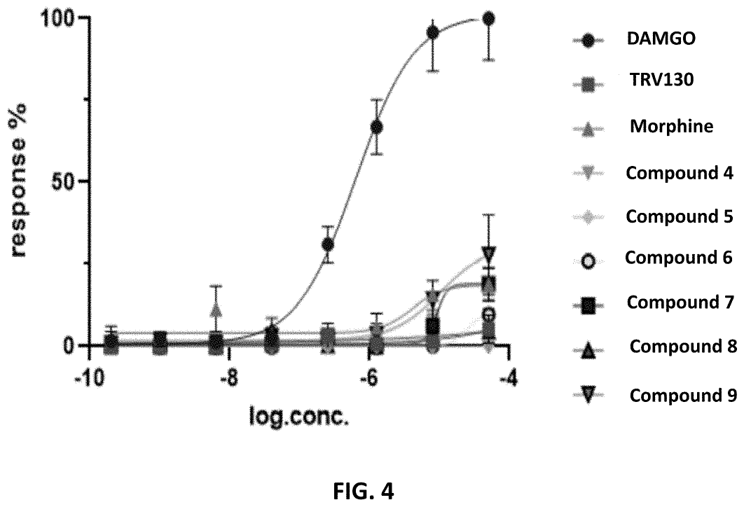

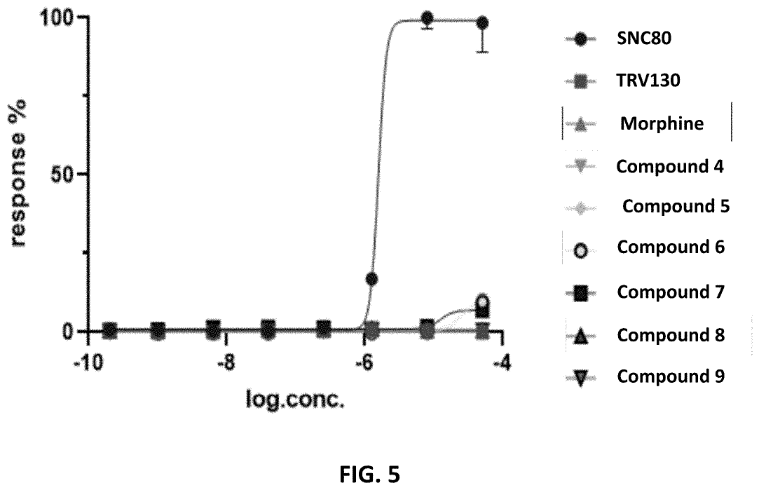

The results of the β-arrestin assay for compounds 4-9 on OPRM and OPRD with morphine are shown in FIG. 4 and FIG. 5. FIG. 4 shows β-arrestin assay result (% bARR2 response) for compounds 4-9 on the μ-Opioid receptor (OPRM) vs a full agonist of OPRM, DAMGO and as compared to morphine. FIG. 5 shows β-arrestin assay result (00 bARR2 response) for compounds 4-9 on the δ-Opioid receptor (OPRD) vs a full agonist of OPRD, SNC80, and as compared to morphine.

### Conclusion

Both cAMP and β-arrestin in vitro assays were conducted with each of the racemic and enantiomer compounds to identify functional selective agonist compounds for the G-protein pathway rather than the β-arrestin pathway on OPRM and OPRD.

In cell lines overexpressing OPRM and OPRD, compounds 5-8 showed Gi efficacy against both receptors.

In the dominant assay (e.g., the experiment for observing the efficacy of Gi after irreversibly lowering the density of the receptor) compounds 6 and 7 derived from compound 2 showed efficacy dominance on both types of opioid receptors.

In the β-arrestin in vitro assay, compounds 4, 6 and 8 exhibited lower bARR2 recruitment as compared to morphine.

### Example 4—Further Investigation of Stereoisomers of Compounds 1-3

As described above, the candidate racemic compounds 1-3 have a comparative advantage in terms of painkiller activity over TRV130, although they have similar pharmacological properties in OPRD.

Compound 2 was demonstrated to be an exemplary candidate compound due to its potency and SNC80, TRV130, PZM21, and Compound 2 produced a full concentration-response curves in dominant assays.

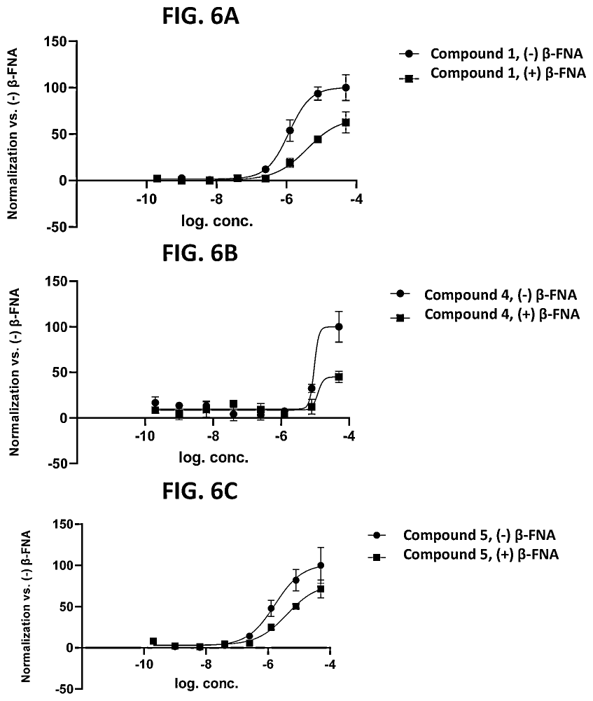

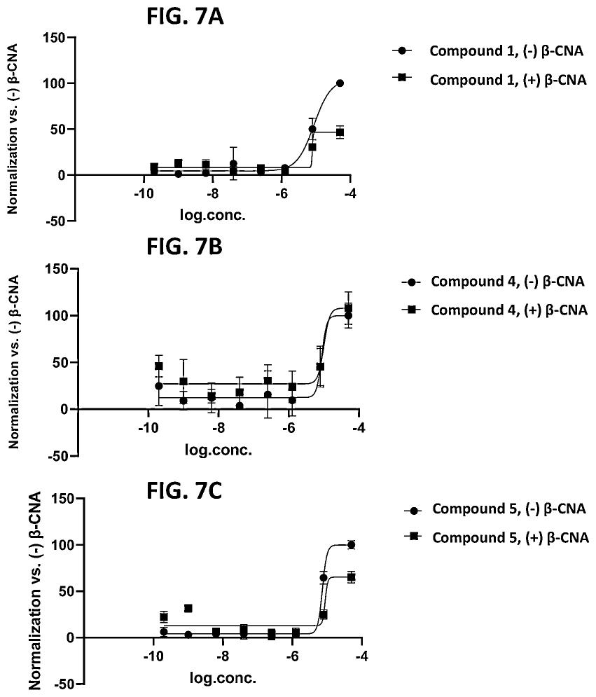

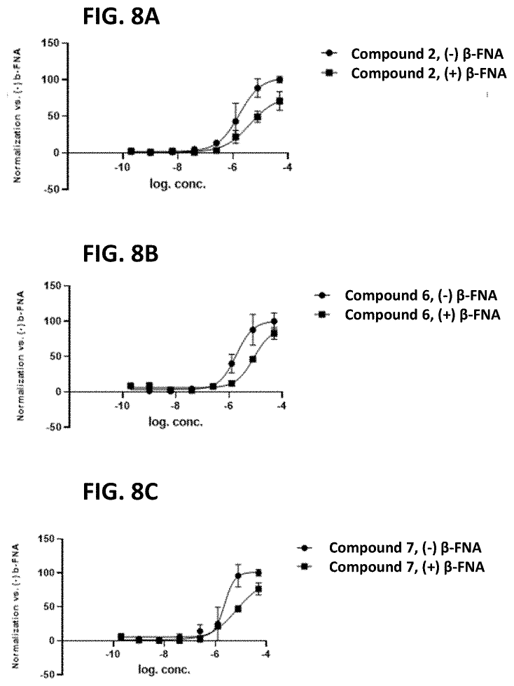

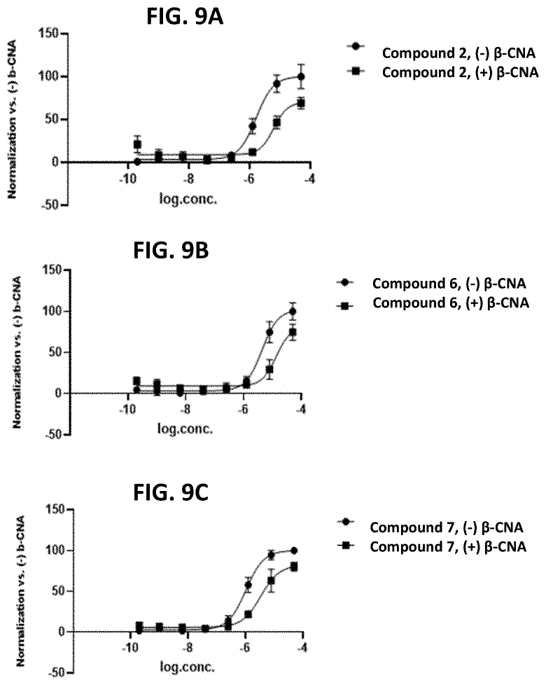

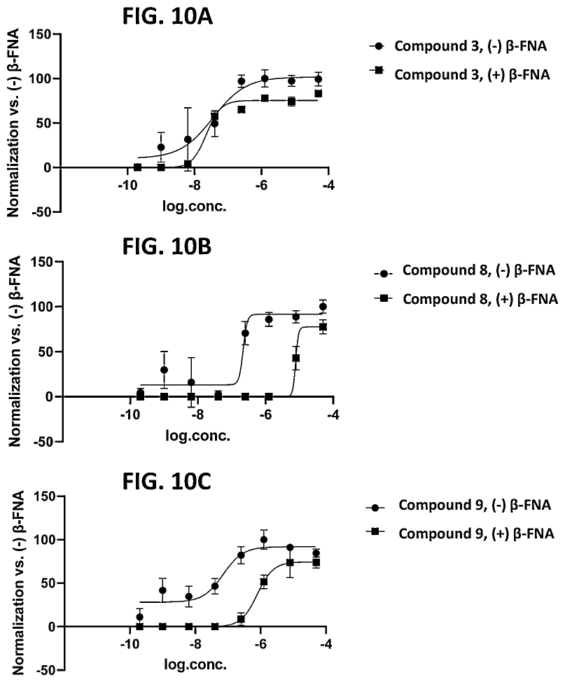

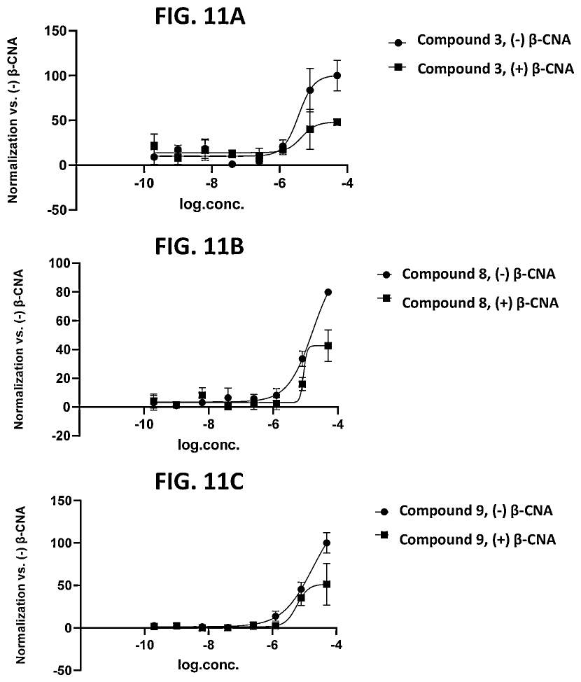

To further investigate the dominant tendency of stereoisomer compounds vs racemic compound, dominant assays were repeated using β-FNA and β-CNA to decrease spare receptors of the OPRM and OPRD cell lines respectively. These results are summarized in Tables 8-10 below, where “β-FNA (−)” refers to no addition of β-FNA and “β-FNA (+) refers to addition of β-FNA; and similarly “β-CNA (−)” refers to no addition of R-CNA and “β-CNA (+) refers to addition of β-CNA. These results are also shown in FIGS. 6A-6C (OPRM, compounds 1, 3 and 5), FIGS. 7A-7C (OPRD, compounds 1, 3 and 5), FIGS. 8A-8C (OPRM, compounds 2, 6 and 7), FIGS. 9A-9C (OPRD, compounds 2, 6 and 7), FIGS. 10A-10C (OPRM, compounds 3, 8 and 9), and FIGS. 11A-11C (OPRD, compounds 3, 8 and 9).

TABLE 8
Dominant assay results for compounds 4 and 5 vs compound 1

| | | | 1 | 4 | 5 |
|---|---|---|---|---|---|
| OPRM | Gi | Eₘₐₓ (%) | 100.3 | 100 | 101.9 |
| | β-FNA (−) | EC₅₀ (nM) | 1161.45 | 9397.23 | 1648.16 |
| | Gi | Eₘₐₓ (%) | 67.51 | 45.02 | 77.98 |
| | β-FNA (+) | EC₅₀ (nM) | 3881.50 | 12022.64 | 4073.80 |
| OPRD | Gi | Eₘₐₓ (%) | 104.5 | 100 | 100 |
| | β-CNA (−) | EC₅₀ (nM) | 8770.01 | 8649.68 | 7328.25 |
| | Gi | Eₘₐₓ (%) | 46.63 | 108.1 | 65.27 |
| | β-CNA (+) | EC₅₀ (nM) | 7870.46 | 10046.16 | 8790.23 |

TABLE 9
Dominant assay results for compounds 6 and 7 vs compound 2

| | | | 2 | 6 | 7 |
|---|---|---|---|---|---|
| OPRM | Gi | Eₘₐₓ (%) | 103 | 100.4 | 100.9 |
| | β-FNA (−) | EC₅₀ (nM) | 1702.16 | 1862.09 | 2306.75 |
| | Gi | Eₘₐₓ (%) | 77.41 | 90.08 | 90.07 |
| | β-FNA (+) | EC₅₀ (nM) | 4187.94 | 8491.80 | 6950.24 |
| OPRD | Gi | Eₘₐₓ (%) | 100.3 | 102.2 | 100.2 |
| | β-CNA (−) | EC₅₀ (nM) | 1648.16 | 4315.19 | 1049.54 |
| | Gi | Emax (%) | 70.18 | 81.41 | 81.99 |
| | β-CNA (+) | EC₅₀ (nM) | 6223.00 | 13489.63 | 3435.58 |

TABLE 10
Dominant assay results for compounds 8 and 9 vs compound 3

| | | | 3 | 8 | 9 |
|---|---|---|---|---|---|
| OPRM | Gi | Eₘₐₓ (%) | 101.8 | 91.58 | 91.98 |
| | β-FNA (−) | EC₅₀ (nM) | 35.97 | 218.78 | 71.29 |
| | Gi | Eₘₐₓ (%) | 75.47 | 77.53 | 74.49 |
| | β-FNA (+) | EC₅₀ (nM) | 22.75 | 7780.37 | 790.68 |
| OPRD | Gi | Eₘₐₓ (%) | 100.4 | 102.7 | 139 |
| | β-CNA (−) | EC₅₀ (nM) | 3630.78 | 16904.41 | 18113.40 |
| | Gi | Eₘₐₓ (%) | 48.23 | 42.67 | 51.61 |
| | β-CNA (+) | EC₅₀ (nM) | 4446.31 | 8689.60 | 5861.38 |

The results indicated that compounds 2, 6 and 7 were all resistant to decreasing spare receptor-induced tendency. This suggested that the maximal responses of all of compounds 2, 6 and 7 were generated from powerful intrinsic efficacy.

FIGS. 8A-8C shows the results from a dominant assay of compounds 2, 6 and 7 using OPRM cell lines. Each of compound 2 (FIG. 8A), compound 6 (FIG. 8B), and compound 7 (FIG. 8C) were resistant to decreasing spare receptors. However, compounds 6 and 7 maintained a higher maximal response as compared to compound 2 despite increased EC₅₀. These results indicate that compounds 6 and 7 exhibit superior efficacy-dominant properties as compared to racemic compound 2 in OPRM cell lines (see e.g., Table 9, β-FNA (+) Emax (%) for compounds 6 and 7 vs compound 2).

FIGS. 9A-9C shows the results from a dominant assay of compounds 2, 6 and 7 using OPRD cell lines. Each of compound 2 (FIG. 9A), compound 6 (FIG. 9B), and compound 7 (FIG. 9C) were resistant to decreasing spare receptors. However, compounds 6 and 7 maintained a higher maximal response as compared to compound 2 despite increased EC₅₀. These results indicate that compounds 6 and 7 also exhibit superior efficacy-dominant properties than racemic compound 2 in OPRD cell lines (see e.g., Table 9, β-CNA (+) Emax (%) for compounds 6 and 7 vs compound 2).

Compound 4 (FIGS. 7A and 7C and Table 8) showed better efficacy than racemic compound 1 in OPRM cell line.

Compound 5 (FIGS. 7A-B and 8A-B and Table 8) showed superior efficacy-dominant properties in OPRM and OPRD cell lines than racemic compound 1.

These results indicate stereoisomer compounds of this disclosure exhibit advanced Gi efficacy over compound racemate.

### EQUIVALENTS AND INCORPORATION BY REFERENCE

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are herein incorporated by reference in their entirety, for all purposes.

1-7. (canceled)

8. A pharmaceutical composition, comprising:
(i) a compound of formula (I): 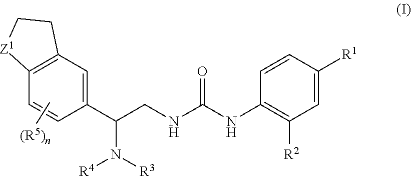
or a stereoisomer thereof, or a pharmaceutically acceptable salt thereof,
wherein:
Z¹ is selected from —O— and —NR⁶—, wherein R⁶ is selected from —H and optionally substituted (C₁-C₃)alkyl;
R¹, R² and each R⁵ are independently selected from H, halogen, OH, (C₁-C₃)alkyl, substituted (C₁-C₃)alkyl, (C₁-C₃)alkoxy, substituted (C₁-C₃)alkoxy; and
R³ and R⁴ are independently selected from —H and optionally substituted (C₁-C₃)alkyl; and
n is 0 to 3; and
(ii) a pharmaceutically acceptable excipient.

9. The pharmaceutical composition of claim 8, wherein Z¹ is —NCH₃—.

10. The pharmaceutical composition of claim 8, wherein:
R¹ is selected from —H and halogen; and
R² is selected from —H and optionally substituted (C₁-C₃)alkyl.

11. The pharmaceutical composition of claim 10, wherein:
R¹ is halogen; and
R² is —H.

12. The pharmaceutical composition of claim 8, wherein R³ and R⁴ are each (C₁-C₃)alkyl.

13. The pharmaceutical composition of claim 12, wherein the compound is 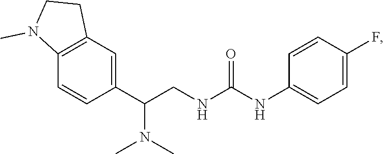 [SMILES: CN1CCc2cc(C(CNC(=O)Nc3ccc(F)cc3)N(C)C)ccc21]
or a stereoisomer thereof, or a pharmaceutically acceptable salt thereof.

14. The pharmaceutical composition of claim 8, wherein Z¹ is —O—.

15. The pharmaceutical composition of claim 14, wherein;
R¹ is selected from —H and halogen; and
R² is selected from —H and optionally substituted (C₁-C₃)alkyl.

16. The pharmaceutical composition of claim 15, wherein:
R¹ is —H; and
R² is optionally substituted (C₁-C₃)alkyl.

17. The pharmaceutical composition of claim 14, wherein R³ and R⁴ are each (C₁-C₃)alkyl.

18. The pharmaceutical composition of claim 17, wherein the compound is 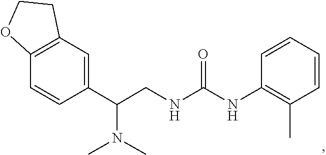 [SMILES: Cc1ccccc1NC(=O)NCC(c1ccc2c(c1)CCO2)N(C)C]
or a stereoisomer thereof, or a pharmaceutically acceptable salt thereof.

19. A pharmaceutical composition, comprising:
(i) a compound of formula (II): 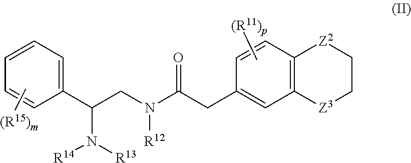
or a stereoisomer thereof, or a pharmaceutically acceptable salt thereof,
wherein:
Z² and Z³ are independently selected from —O— and —NR¹⁶—, wherein R¹⁶ is selected from —H and optionally substituted (C₁-C₃)alkyl;
R¹², R¹³ and R¹⁴ are independently selected from —H and optionally substituted (C₁-C₃)alkyl;
each R¹¹ and each R¹⁵ are independently selected from —H, -halogen, —OH, —CF₃, —OCF₃, —CN, —NH₂, —NO₂, optionally substituted (C₁-C₆)alkyl, optionally substituted (C₁-C₆)alkoxy, —C(O)OR¹², —C(O)NHR¹², —SO₂NHR¹², —NR¹³R¹⁴, —NHC(O)R¹², and —SO₃H;
m is 0 to 5; and
p is 0 to 3; and
(ii) a pharmaceutically acceptable excipient.

20. The pharmaceutical composition of claim 19, wherein R¹² is —H.

21. The pharmaceutical composition of claim 20, wherein the compound is of formula (III): 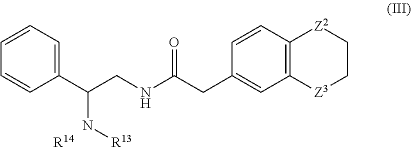
or a stereoisomer thereof, or a pharmaceutically acceptable salt thereof.

22. The pharmaceutical composition of claim 21, wherein Z² is —O—.

23. The pharmaceutical composition of claim 22, wherein Z³ is —O—.

24. The pharmaceutical composition of claim 21, wherein R¹³ and R¹⁴ are each (C₁-C₃)alkyl.

25. The pharmaceutical composition of claim 21, wherein the compound is 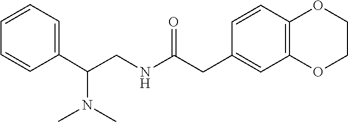 [SMILES: CN(C)C(CNC(=O)Cc1ccc2c(c1)OCCO2)c1ccccc1]
or a stereoisomer thereof, or a pharmaceutically acceptable salt thereof.

26. A method of modulating the activity of an opioid receptor, comprising contacting a biological system with a a pharmaceutical composition comprising
(i) a compound of formula (I) or formula (II): 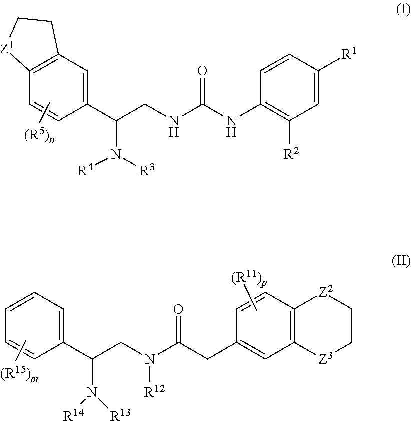
or a stereoisomer thereof, or a pharmaceutically acceptable salt thereof,
wherein:
Z¹ is selected from —O— and —NR⁶—, wherein R⁶ is selected from —H and optionally substituted (C₁-C₃)alkyl;
R¹, R² and each R⁵ are independently selected from H, halogen, OH, (C₁-C₃)alkyl, substituted (C₁-C₃)alkyl, (C₁-C₃)alkoxy, substituted (C₁-C₃)alkoxy;
R³ and R⁴ are independently selected from —H and optionally substituted (C₁-C₃)alkyl;
n is 0 to 3;
Z² and Z³ are independently selected from —O— and —NR¹⁶—, wherein R¹⁶ is selected from —H and optionally substituted (C₁-C₃)alkyl;
R¹², R¹³ and R¹⁴ are independently selected from —H and optionally substituted (C₁-C₃)alkyl;
each R¹¹ and each R¹⁵ are independently selected from —H, -halogen, —OH, —CF₃, —OCF₃, —CN, —NH₂, —NO₂, optionally substituted (C₁-C₆)alkyl, optionally substituted (C₁-C₆)alkoxy, —C(O)OR¹², —C(O)NHR¹², —SO₂NHR¹², —NR¹³R¹⁴, —NHC(O)R¹², and —SO₃H;
m is 0 to 5; and
p is 0 to 3; and
(ii) a pharmaceutically acceptable excipient.

27. The method of claim 26, wherein the biological system is in vitro.

28. The method of claim 26, wherein the biological system is in vivo.

29. A method of treating pain, comprising administering to a subject having pain an effective amount of a a pharmaceutical composition to treat the subject for pain, wherein the pharmaceutical composition comprises:
(i) a compound of formula (I) or formula (II): 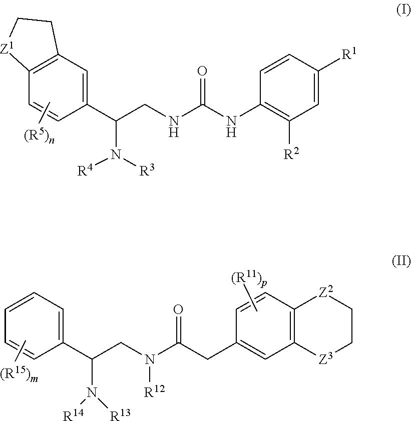
or a stereoisomer thereof, or a pharmaceutically acceptable salt thereof,
wherein:
Z¹ is selected from —O— and —NR⁶—, wherein R⁶ is selected from —H and optionally substituted (C₁-C₃)alkyl;
R¹, R² and each R⁵ are independently selected from H, halogen, OH, (C₁-C₃)alkyl, substituted (C₁-C₃)alkyl, (C₁-C₃)alkoxy, substituted (C₁-C₃)alkoxy;
R³ and R⁴ are independently selected from —H and optionally substituted (C₁-C₃)alkyl;
n is 0 to 3;
Z² and Z³ are independently selected from —O— and —NR¹⁶—, wherein R¹⁶ is selected from —H and optionally substituted (C₁-C₃)alkyl;
R¹², R¹³ and R¹⁴ are independently selected from —H and optionally substituted (C₁-C₃)alkyl;
each R¹¹ and each R¹⁵ are independently selected from —H, -halogen, —OH, —CF₃, —OCF₃, —CN, —NH₂, —NO₂, optionally substituted (C₁-C₆)alkyl, optionally substituted (C₁-C₆)alkoxy, —C(O)OR¹², —C(O)NHR¹², —SO₂NR¹², —NR¹³R¹⁴, —NHC(O)R¹², and —SO₃H;
m is 0 to 5; and
p is 0 to 3; and
(ii) a pharmaceutically acceptable excipient.

30. The method of claim 29, wherein the compound is 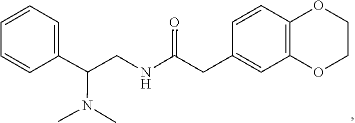 [SMILES: CN(C)C(CNC(=O)Cc1ccc2c(c1)OCCO2)c1ccccc1]
or a stereoisomer thereof, or a pharmaceutically acceptable salt thereof.

31. The method of claim 29, wherein the compound is 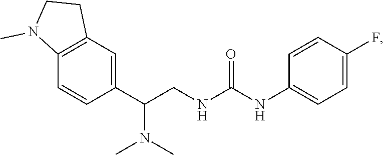 [SMILES: CN1CCc2cc(C(CNC(=O)Nc3ccc(F)cc3)N(C)C)ccc21]
or a stereoisomer thereof, or a pharmaceutically acceptable salt thereof.

32. The method of claim 29, wherein the compound is 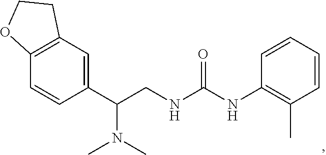 [SMILES: Cc1ccccc1NC(=O)NCC(c1ccc2c(c1)CCO2)N(C)C]
or a stereoisomer thereof, or a pharmaceutically acceptable salt thereof.

33-35. (canceled)
